SEQUENCE LISTING (20 sequences)

SEQ ID NO: 1 - DNA, 30 bp, Artificial Sequence
  Synthetic Construct
gatccaattgcctaactctttcgaatactt

SEQ ID NO: 2 - DNA, 30 bp, Artificial Sequence
  Synthetic Construct
gatcgctagcggaagagtcattcaccatac

SEQ ID NO: 3 - DNA, 30 bp, Artificial Sequence
  Synthetic Construct
gatcgcgcgcgtacatacatcgccgtcatc

SEQ ID NO: 4 - DNA, 31 bp, Artificial Sequence
  Synthetic Construct
gatccagctgttatggaggatatgtgaacgc

SEQ ID NO: 5 - DNA, 30 bp, Artificial Sequence
  Synthetic Construct
gatccaattgtgacgtacatgagtctgagt

SEQ ID NO: 6 - DNA, 30 bp, Artificial Sequence
  Synthetic Construct
gatcgctagccgtggatatgatgattctct

SEQ ID NO: 7 - DNA, 30 bp, Artificial Sequence
  Synthetic Construct
gatcgcgcgctttgcatgttaccactatca

SEQ ID NO: 8 - DNA, 30 bp, Artificial Sequence
  Synthetic Construct
gatccagctgcagacatggatctgtcacga

SEQ ID NO: 9 - DNA, 30 bp, Artificial Sequence
  Synthetic Construct
gatccaattgtaccggatctacgttctact

SEQ ID NO: 10 - DNA, 30 bp, Artificial Sequence
  Synthetic Construct
gatcgctagcggatatatagatgtcaatta

SEQ ID NO: 11 - DNA, 28 bp, Artificial Sequence
  Synthetic Construct
gatcgcgcgcataatccttctcgtatac

SEQ ID NO: 12 - DNA, 30 bp, Artificial Sequence
  Synthetic Construct
gatccagctgtgctgatcctcccattccgt

SEQ ID NO: 13 - DNA, 30 bp, Artificial Sequence
  Synthetic Construct
gatccaattgcacgataatatcagaggagt

SEQ ID NO: 14 - DNA, 30 bp, Artificial Sequence
  Synthetic Construct
gatcgctagccttcattacgtatgactaat

SEQ ID NO: 15 - DNA, 30 bp, Artificial Sequence
  Synthetic Construct
gatcgcgcgctgacttacttgtataataag

SEQ ID NO: 16 - DNA, 30 bp, Artificial Sequence
  Synthetic Construct
gatccagctgcagaacatgtagacgaatca

SEQ ID NO: 17 - DNA, 30 bp, Artificial Sequence
  Synthetic Construct
gatccaattgcgggagacgaggatatagct

SEQ ID NO: 18 - DNA, 36 bp, Artificial Sequence
  Synthetic Construct
gatccccgggaggcctatagacctctgtacataaaa

SEQ ID NO: 19 - DNA, 36 bp, Artificial Sequence
  Synthetic Construct
gatccccgggacgcgtgacaatgatgcggaagaaca

SEQ ID NO: 20 - DNA, 36 bp, Artificial Sequence
  Synthetic Construct
gatcgacttgagcgtcatctggtagatagaccatcg

(19) United States
(12) Patent Application Publication
(10) Pub. No.: US 2020/0046784 A1
(43) Pub. Date: Feb. 13, 2020

(54) RECOMBINANT ONCOLYTIC VIRUSES FOR CANCER THERAPY

(71) Applicants: UNIVERSITY HEALTH NETWORK, TORONTO (CA); CHILDREN'S HOSPITAL OF EASTERN ONTARIO RESEARCH INSTITUTE INC., OTTAWA (CA)

(72) Inventors: Judith Andrea MCCART, TORONTO (CA); David Francis STOJDL, OTTAWA (CA); Tiffany Yun-Yee HO, MISSISSAUGA (CA)

(21) Appl. No.: 16/338,263

(22) PCT Filed: Oct. 2, 2017
(86) PCT No.: PCT/CA2017/051171

§ 371 Date: Mar. 29, 2019

Related U.S. Application Data
(60) Provisional application No. 62/402,574, filed on Sep. 30, 2016.

(51) Int. Cl. A61K 35/768; A61P 35/00; C12N 15/86; A61P 31/00

(52) U.S. Cl. CPC A61K 35/768; A61P 31/00; C12N 15/86; A61P 35/00

(57) ABSTRACT

The disclosure provides a recombinant oncolytic poxvirus including vaccinia virus comprising one or more inactivated immunomodulatory gene selected from NIL, K1L, K3L, A46R, and/or A52R, optionally further comprising inactivated TK and/or VGF genes. The disclosure also provides methods and for the use of these recombinant oncolytic poxvirus in oncolytic virotherapy. Also provided in the disclosure are vector constructs for generating recombinant oncolytic poxviruses including for example vaccinia viruses that have one or more inactivated immunomodulatory genes.

### RELATED PATENT APPLICATION

This application claims the benefit or priority of U.S. Provisional Application No. 62/402,574 filed on Sep. 30, 2016, the content of which is incorporated herein in its entirety, including drawings.

### FIELD

This disclosure generally relates to the field of oncology and oncolytic viruses. More particularly, it concerns compositions and methods of treating cancer in a patient using oncolytic poxviruses deleted of immunomodulatory genes.

### BACKGROUND

Chemotherapy and radiotherapy are often used as a non-surgical means to treat many malignancies today. However, these treatments are considerably non-specific and have many detrimental side effects. Oncolytic virotherapy uses replication-competent viruses to kill tumour cells while leaving normal cells unscathed. Several viruses have been examined as potential virotherapy agents such as Newcastle disease virus (NDV), reovirus (RV), measles virus (MV), herpes simplex virus (HSV), vesicular stomatitis virus (VSV), and poxviruses such as vaccinia virus (VV). Some viruses usually replicate in non-human hosts but are found to be naturally oncotropic (e.g. NDV, RV) while other oncolytic viral vectors are tumour-selective based on engineered attributes (e.g. VV, VSV, MV) (Antonio Chiocca 2002).

The basic concept and prior art in the field of oncolytic virus has been described, for examples, in U.S. Pat. Nos. 8,778,328, 8,980,246, 8,986,674, and 9,005,602. These documents are incorporated herein by reference.

U.S. Pat. No. 8,778,328 relates a poxvirus comprising a defective F4L and/or I4L gene and compositions involving such poxvirus useful for the treatment of cancer.

U.S. Pat. No. 8,980,246 relates to methods that include a thymidine kinase deficient vaccinia virus. The methods include administering the vaccinia virus at increased viral concentrations.

U.S. Pat. No. 8,986,674 relates to methods and compositions for the treatment of cancer and cancer cells using altered poxviruses, including a vaccinia virus that has been altered to generate a more effective therapeutic agent. The methods and compositions involve poxviruses that possess mutations that result in poxviruses with diminished or eliminated capability to implement an antiviral response in a host.

U.S. Pat. No. 9,005,602 relates to viruses and methods for preparing modified vaccinia viruses. The modified viruses can be used in methods of treatment of diseases, such as proliferative and inflammatory disorders, including cancer, and as anti-tumour and/or antiangiogenic agents.

Currently, the safety of oncolytic VVs in clinical development, such as double deleted VV (vvDD), a Western Reserve vaccinia virus with two non-essential gene deletions from its genome (i.e. deleted thymidine kinase (TK) and vaccinia growth factor (VGF); McCart et al., 2001), is enhanced by improving the inherent ability of the virus to selectively infect and kill fast-dividing cells, such as tumours.

Dosages used in oncolytic virus treatment against tumours are typically found in the 10⁸-10⁹ pfu range. In a patient with two large melanoma lesions, intratumoural treatment with 3×10⁹ PFU vvDD in one lesion resulted in viral replication in both injected and non-injected masses, yet the 1 mm strip of normal skin between them was spared (Zeh et al., 2015). In a trial, a randomized trial with JX-594 (Wyeth strain VV with deletion in thymidine kinase, expressing GM-CSF), overall survival was 14.1 months compared to 6.7 months between advanced hepatocellular carcinoma patients treated intratumourally with 1×10⁹ pfu compared to 1×10⁸ pfu, respectively (Heo et al., 2013), suggesting that higher viral dosage results in improved anti-tumour response.

Alternate or improved oncolytic viruses are desirable.

### SUMMARY

More potent but equally safe VVs may be generated by exploiting the dampened ability to respond to virus infections in many tumours.

In the present disclosure, a deletion in poxvirus immunomodulatory genes (i.e., N1L, K1L, K3L, A46R, or A52R) yields oncolytic poxviruses such as VVs, against cancers such as colon and ovarian cancer. As described in the present disclosure, imparting tumour-selectivity via this strategy increases oncolytic virus potency by generating safe and effective VVs for cancer therapy that could be used at lower doses than clinical candidate vvDD.

Recombinant oncolytic poxvirus' comprising an inactivated immunomodulatory gene, including N1L, K1L, K3L, A46R and A52R, are generated and isolated. The disclosure demonstrates that poxviruses such as vaccinia viruses (VVs) with a deletion of an above said immunomodulatory gene have similar or improved in vitro replication, spread, and/or cytotoxicity in colon cancer cells compared to vvDD and exhibit oncolytic activity against ovarian cancer cells. Remarkably, several VV deletion mutants (ΔK1L VV, ΔA46R VV, and ΔA52R VV) demonstrated tumour-selectivity and prolonged tumour-survival at doses 20-1000 times lower than treatment dose with clinical candidate vvDD (Western Reserve VV with deletions in TK and VGF).

Accordingly, the present disclosure provides a recombinant vector comprising:
a) a left flanking portion of a poxvirus gene selected from N1L, K1L, K3L, A46R, and A52R gene, and
b) a right flanking portion of said poxvirus gene,
wherein the left flanking portion and the right flanking portion are operably linked to a detectable interrupter expression cassette.

In a preferred embodiment, the poxvirus gene is selected from K1L, A46R and A52R.

In one embodiment, the detectable interrupter expression cassette directs the expression of one or more polypeptides, optionally a fluorescent protein, a luciferase enzyme or a xanthine-guanine phosphoribosyltransferase (gpt) protein.

In another embodiment, the recombinant vector is a shuttle plasmid, optionally comprising one or more fluorescent proteins, luciferase enzymes or gpt proteins, wherein the poxvirus gene is selected from N1L, K1L, K3L, A46R or A52R, preferably K1L, A46R or A52R.

In an embodiment, each flanking portion of a poxvirus gene comprises about 200-600 nucleotide residues, preferably about 300-500 nucleotide residues, of flanking sequence.

In another embodiment, the flanking sequence has at least 80%, at least 85%, at least 90%, or at least 95% sequence identity to corresponding sequence in a poxvirus, optionally Accession number: NC_006998.1.

In an embodiment, the recombinant oncolytic poxvirus or viral DNA thereof was constructed using the recombinant vector as described herein.

In another embodiment, a cell is transfected with a recombinant vector described herein, optionally infected with a poxvirus, preferably a vaccinia virus.

In an embodiment, a recombinant oncolytic poxvirus, optionally a recombinant oncolytic vaccinia virus or viral DNA thereof, comprising one or more inactivated poxvirus genes selected from N1L, K1L, K3L, A46R, and/or A52R, preferably selected from K1L, A46R and/or A52R, optionally further comprising an inactivated TK gene and/or one or more inactivated growth factor genes, optionally VGF genes.

In an embodiment, a poxvirus gene, optionally an immunomodulatory gene selected from N1L, K1L, K3L, A46R and A52R, is inactivated by one or more mutations, optionally deletion mutation comprising deletion of all or a portion of the gene.

In another embodiment, an inactivated poxvirus gene is inactivated by replacing the poxvirus genes with a detectable interrupter expression cassette, optionally the detectable interrupter expression cassette comprises one or more fluorescent proteins, luciferase enzymes or gpt proteins.

In an embodiment, a recombinant oncolytic poxvirus or viral DNA thereof described herein is constructed using any of the recombinant vectors described herein.

In another embodiment, the recombinant oncolytic poxvirus or viral DNA thereof is constructed using a parental strain selected from Lister, Wyeth, modified vaccinia Ankara, CV-1, Western Reserve, Copenhagen, Tian Tian, NYCBH, Lister, modified vaccinia Ankara, Lederle, Temple of Heaven, Tashkent, USSR, Evans, Praha, LIVP, Ikeda, IHD, IHD-W, DPP25, IOC, Dls, LC16, EM-63, IC, Malbran, DUKE, Acambis, 3737, CVA, VJS6 and AS.

In an embodiment, a cell is infected an oncolytic recombinant poxvirus or comprising oncolytic viral DNA described herein.

In another embodiment, the cell is a tumour cell, optionally an ovarian cancer cell, a colorectal cancer cell, a hepatocellular carcinoma cell, a lung cancer cell, a mesothelioma cell, a prostate cancer cell, a melanoma cell, a renal cell carcinoma cell, a head and neck cancer cell, a pancreatic cancer cell, a glioma cell, a gastric cancer cell and/or a breast cancer cell.

In an embodiment, a composition comprises a recombinant vector, cell or recombinant oncolytic poxvirus or viral DNA thereof described herein, and a suitable diluent, optionally a pharmaceutically suitable diluent.

In another embodiment, a composition comprises a recombinant oncolytic poxvirus described herein, optionally a recombinant oncolytic poxvirus, optionally a recombinant vaccinia virus, wherein concentration of the recombinant oncolytic poxvirus is between 1×10⁶ and 5×10⁷ pfu/dose, optionally 1×10⁶, 1×10⁷, or 5×10⁷ pfu/dose.

The present disclosure also provides an in vitro method of making any recombinant oncolytic poxvirus or viral DNA thereof described herein, comprising
a) introducing a recombinant vector described herein into cells infected with a poxvirus, optionally vaccinia virus, under conditions suitable for recombination between the recombinant vector and the recombinant oncolytic poxvirus or viral DNA thereof; and
b) isolating the oncolytic poxvirus or viral DNA thereof inactivated for the poxvirus gene selected from N1L, K1L, K3L, A46R, and A52R, preferably K1L, A46R and A52R.

The present disclosure further provides a method of killing cancer cells, comprising contacting the cancer cells or administering to a subject with a cancer or a tumour comprising cancer cells, an effective amount of a recombinant oncolytic poxvirus or viral DNA thereof described herein. Also provided is use of an effective amount of a recombinant oncolytic poxvirus or viral DNA thereof described herein for killing cancer cells. Also provided is use of an effective amount of a recombinant oncolytic poxvirus or viral DNA thereof described herein in the manufacture of a medicament for treating a subject with cancer. Even further provided is an effective amount of a recombinant oncolytic poxvirus or viral DNA thereof described herein for use in treating a subject with cancer.

In an embodiment, the cancer cells are solid tumour cells, optionally selected from ovarian cancer cells, colon cancer cells, hepatocellular carcinoma cells, lung cancer cells, mesothelioma cells, prostate cancer cells, melanoma cells, renal cell carcinoma cells, head and neck cancer cells, pancreatic cancer cells, glioma cells, gastric cancer cells and breast cancer cells, wherein the cancer is a blood cancer, optionally myeloma or leukemia, or wherein the cancer is a blood cell tumour, optionally lymphoma.

In another embodiment, the subject has late stage cancer, optionally having peritoneal carcinomatosis (PC).

The present disclosure also provides an isolated DNA molecule used for amplifying flanking regions of a poxvirus gene selected from N1L, K1L, K3L, A46R and A52R, optionally the isolated DNA molecule selected from SEQ ID NOs: 1-20.

In an embodiment, an isolated DNA molecule comprises 5′ and/or 3′ overhang and sequence of flanking portion of a poxvirus gene selected from N1L, K1L, K3L, A46R and A52R, wherein the sequence amplified using a primer pair, optionally the primer pair selected from SEQ ID NOs: 1-20.

In one embodiment, a pharmaceutical composition comprising a recombinant oncolytic poxvirus, optionally a recombinant vaccinia virus, wherein the recombinant poxvirus comprises one of more inactivated poxvirus genes selected from N1L, K1L, K3L, A46R and A52R, preferably selected from K1L, A46R and A52R, wherein concentration of the recombinant oncolytic poxvirus is between 1×10⁶ and 5×10⁷ pfu/dose, optionally 1×10⁶, 1×10⁷, or 5×10⁷ pfu/dose.

In another embodiment, a pharmaceutical composition comprising a recombinant oncolytic poxvirus, optionally a recombinant vaccinia virus, for the treatment of cancer in a subject, wherein the cancer is a solid tumour cancer, optionally selected from ovarian cancer, colon cancer, hepatocellular carcinomas, lung cancer, mesothelioma, prostate cancer, melanoma, renal cell carcinoma, head and neck cancer, pancreatic cancer, glioma, gastric cancer, and breast cancer cells, wherein the cancer is a blood cancer, optionally myeloma or leukemia, or wherein the cancer is a blood cell tumour, optionally lymphoma.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1 shows a schematic diagram of shuttle plasmid and final construct of deletion VV candidate using ΔN1L VV as an example. A) Diagram of the shuttle plasmid created to facilitate homologous recombination with VV to generate deletion mutant interrupted with RFP/Rluc and gpt genes as illustrated in B). RFP and Rluc are conjoined by a foot-and-mouth disease virus 2A motif. Abbreviations: RFP: red fluorescent protein, Rluc: Renilla luciferase, PSyn/late: synthetic VV early/late promoter, p7.5: p7.5 VV early/late promoter, xgprt: xanthine-guanine phosphoribosyltransferase.

FIG. 2 shows confirmation of pVX-R2R-LUC plasmid. Existing DNA plasmid preparations were confirmed to be pVX-R2R-LUC by 1) a double digest with SpeI and MfeI, which yielded the expected band sizes of 6 kb and 800 bp and 2) a double digest with BssHII and DrdI, which yielded the expected band size of two 3.2 kb and one 1 kb bands.

FIG. 3 shows left and right flanking DNA fragments of VV N1L gene as generated by PCR. Primers to amplify the DNA sequences flanking the candidate VV gene, N1L, were designed from the WR VV genome sequence. PCR products amplified from the VJS6 viral DNA were confirmed by agarose gel. A) Left-flanking DNA, N1L-L, and B) Right-flanking DNA, N1L-R. Neg=negative control (H₂O).

FIG. 4 shows confirmation of pVX-R2R-Luc with left-flanking DNA insertion. A) Half a colony of ampicillin-selected transformants were directly confirmed to contain the left-flanking DNA insert (N1L-L) by colony PCR Positive control: VJS6 DNA. Negative control (Neg): H2O. At least 3 clones were tested for the presence of the desired partial shuttle plasmid. B) Plasmid DNA preparations were confirmed to contain the desired left side flanking DNA fragment (N1L-L) by a restriction enzyme digest based on a unique or rare cut sites within the insert and in the pVX-R2R-Luc. Specifically, potential pVX-N1L-L-R2R-Luc plasmid DNA was digested with HindIII (expected sizes: ˜3 kb and ˜4.5 kb).

FIG. 5 shows confirmation of the final shuttle plasmid. A) Half a colony of each transformant was directly used for colony PCR confirmation for the presence of the right-flanking DNA fragment, N1L-R. Negative control (Neg): H₂O. B) Plasmid DNA preparations from positive clones in A) were confirmed to contain the desired right side flanking DNA fragment by a restriction enzyme digest based on a unique or rare cut sites within the insert and in the pVX-R2R-Luc. pVX-N1L-R2R-Luc DNA from 2 clones digested with HindIII (expected sizes: ˜800 bp and ˜5.3 kb).

FIG. 6 shows sample images of CV-1 cells after transfection/infection and subsequent rounds of re-infection with recombinant VV. CV-1 cells were transfected with shuttle plasmid, infected with wildtype WR VV (F13L+), then incubated in media containing mycophenolic acid, hypoxanthine, and xanthine (drugged DMEM). The transfected/infected monolayer was harvested 72 hpi and re-infected into another CV-1 monolayer grown in drugged DMEM (Round 0). Subsequent RFP plaques were harvested and re-infected into new monolayers for at least 5 more rounds. Brightfield and fluorescent images (Cy3 filter) were taken with a 10× objective lens at 72 hpi after each round.

FIG. 7 shows PCR confirmation of the absence of parental virus from recombinant VV plaques. CV-1 cells were infected by one of 6 plaques after round 5 of mycophenolic acid selection. When complete cytopathic effect (CPE) was observed, cells were harvested and digested with Proteinase K. The samples were tested with PCR for the absence of parental virus (F13L+). The forward primer from the left flanking DNA fragment and the reverse primer of the right flanking DNA fragment were used as primers for the PCR reactions. Positive control for parental virus was viral DNA from F13L+ infected CV-1 cells, and the corresponding shuttle plasmid DNA was used as template for the positive control for the recombinant band, negative (Neg) control was mock-infected cells.

FIG. 8 shows PCR confirmation of candidate viral stocks. CV-1 cells were infected with virus stocks amplified from plaques of candidate VVs. Infected cells were harvested and PCR with the forward primer of the left flanking insert and the reverse primer of the right flanking insert was conducted to amplify the corresponding wildtype candidate VV gene (˜1 kb) or a PCR product reflective of the marker genes interrupting the candidate genes (˜4 kb). Negative control (Neg) was mock-infected cells, F13L+(wildtype WR VV)-infected cells were positive control for the wildtype product, and the corresponding shuttle plasmids, if available, was the positive control for the PCR product expected from the candidate deletion viruses: A) ΔN1L VV, B) ΔK1L VV, C) ΔK3L VV, D) ΔA46R VV, and E) ΔA52R VV.

FIG. 9 shows viral replication of candidate VVs and vvDD in monolayers of colon and ovarian cancer cell lines. Monolayers of MC38 (A, B), DLD-1 (C, D), and A2780 (E, F) cells were infected with vvDD or candidate VVs at a multiplicity of infection (MOI) of 0.1. Viral quantities were determined by plaque assays at 2, 24, 48, and 72 hpi. Data are presented as total plaque-forming units (pfu) over time (A, C, E) or as fold change relative to baseline pfu at 2 hpi (B, D, F). Data presented as Mean±SEM (n=3). *p<0.05 compared to vvDD.

FIG. 10 shows representative images of viral spread of VV deletion mutants and vvDD in monolayers of colon and ovarian carcinoma cell lines. Cell lines MC38, DLD-1, and A2780 were infected with either the VV deletion mutants or vvDD at an MOI of 0.1 and visualized under fluorescent microscopy with a Cy3 lens at 10× magnification. Notable differences in RFP expression were seen at 72 hpi in MC38 and DLD-1 cells and at 48 hpi in A2780 cells.

FIG. 11 shows quantification of viral spread in monolayers of colon and ovarian carcinoma cells. Monolayers of A) MC38, B) DLD-1, and C) A2780 cells were infected by candidate VVs and vvDD at an MOI of 0.1. Fluorescent images were taken with a Cy3 lens at 10× magnification and infected area was calculated by measuring area with RFP marker expression over total image area as calculated with ImageJ software set at a uniform threshold at each time point. Data are a combination of two independent experiments.

FIG. 12 shows cytotoxicity of vvDD and candidate VVs towards colon and ovarian cancer cells. Monolayers of MC38 (A, D, G), DLD-1 (B, E, H), and A2780 (C, F, I) were infected at an MOI of A-C) 0.1, D-F) 1, and G-I) 5, and cell viability was measured at 2, 24, 48, and 72 hpi. Data are presented as Mean±SEM (n=3). *p<0.05 compared to vvDD (black square).

FIG. 13 shows representative images of viral spread of VV deletion mutants and vvDD in MC38 tumour spheroids. MC38 spheroids were infected at an MOI of 2 and visualized with confocal microscopy with brightfield and Cy3 lens at 10× objective lens (n=4). Scale bar=100 μm. Top panel: Brightfield picture of spheroids before infection. Middle pane: Brightfield pictures of MC38 spheroids 72 hpi. Bottom panel: Virally-induced RFP expression at a cross-section of spheroids at 150 μm (approximately halfway) visualized with a Cy3 lens.

FIG. 14 shows representative images of viral spread of VV deletion mutants and vvDD in DLD-1 tumour spheroids. DLD-1 spheroids were infected at an MOI of 2 and visualized with confocal microscopy with brightfield and Cy3 filter at 10× objective lens 72 hpi (n=4). Scale bar=100 μm. Top panel: Brightfield images of spheroids before infection. Middle panel: Brightfield images of DLD-1 spheroids at 72 hpi. Bottom panel: Virally-induced RFP expression at a cross-section of spheroids at 150 m (approximately halfway) visualized with a Cy3 filter.

FIG. 15 shows MC38 spheroid size after treatment with candidate VVs or vvDD. MC38 spheroids were infected at an MOI of 2 and brightfield images were taken at 24 hour intervals post-infection with a 10× objective lens. Tumour volume was calculated based on the radii at the major and minor axis of the spheroid measured with the images. V=4/3*pi*(minor axis)²(major axis). Data are presented as Mean±SEM (n=4).

FIG. 16 shows a clonogenic assay of tumour spheroids treated with vvDD or a candidate VV. A) MC38 and B) DLD-1 spheroids infected at MOI 2 were disassociated at 96 hpi and reseeded into 6-well plates at 25-100 cells/well, grown for 10 days, and stained with crystal violet. Surviving fraction was calculated based on the number of colonies (>50 cells) relative to the number of cells originally seeded.

FIG. 17 shows a schematic of double deleted VV (vvDD) and replication in various tissue types.

FIG. 18 shows a schematic of N1L recombinant virus and parental virus.

FIG. 19 shows intraperitoneal maximum tolerable doses (MTD) of candidate VVs in C57BL/6 mice. Non-tumour-bearing mice were injected IP at indicated doses with candidate VV or vvDD. A) 5×10⁷ pfu into C57BL/6 mice, and B) 1×10⁷ pfu into C57BL/6 mice.

FIG. 20 shows intraperitoneal maximum tolerable doses (MTD) of candidate VVs in nude (NU/NU) mice. Non-tumour-bearing mice were injected IP with 1×10⁶ pfu candidate VV or vvDD into NU/NU mice.

FIG. 21 shows biodistribution and VV tumour-selectivity of viral replication of recombinant VVs in C57BL/6 mice with MC38 tumours. C57BL/6 mice were injected IP with MC38 tumour cells then treated with HBSS alone, vvDD, or a candidate VV deletion mutant 12 days later. Tumour and non-tumour tissues were harvested 6 days post-infection (n=3) and viral load was quantified by plaque assay. Dose: vvDD=1×10⁹ pfu; ΔK1L VV=5×10⁷ pfu; ΔA46R VV=1×10⁷ pfu; ΔA52R=1×10⁷ pfu. Viral load was quantified and presented as total pfu/mg. Values are means of triplicates±SEM.

FIG. 22 shows biodistribution and VV tumour-selectivity of viral replication of recombinant VVs in NU/NU mice with DLD-1 tumours. NU/NU mice were injected IP with DLD-1 tumour cells then treated with HBSS alone, vvDD, or a candidate VV deletion mutant 12 days later. Tumour and non-tumour tissues were harvested 6 days post-infection (n=3) and viral load was quantified by plaque assay. Dose: all VVs=5×10⁶ PFU. Viral load was quantified and presented as total pfu/mg. Values are means of triplicates±SEM.

FIG. 23 shows biodistribution and VV tumour-selectivity of viral replication of recombinant VVs in NU/NU mice with A2780 tumours. NU/NU mice were injected IP with A2780 tumour cells then treated with HBSS alone, vvDD, or a candidate VV deletion mutant 12 days later. Tumour and non-tumour tissues were harvested 6 days post-infection (n=3) and viral load was quantified by plaque assay. Dose: all VVs=5×10⁶ pfu. Viral load was quantified and presented as total pfu/mg. Values are means of triplicates±SEM.

FIG. 24 shows VV tumour-efficacy. C57BL/6 mice were injected IP with MC38 tumour cells then treated with HBSS alone, vvDD, or a candidate VV deletion mutant (n=8) 12 days later and followed for survival. Kaplan-Meier curves of MC38-bearing C57BL/6 mice were plotted (dose: vvDD=1×10⁹ pfu; ΔK1LVV=5×10⁷ pfu; ΔA46R VV=1×10⁷ pfu; ΔA52R=1×10⁷ PFU). Statistical significance was calculated by log-rank test.

FIG. 25 shows VV tumour-efficacy. NU/NU mice were injected IP with DLD-1 tumour cells then treated with HBSS alone, vvDD, or a candidate VV deletion mutant (n=8) 12 days later and followed for survival. Kaplan-Meier curves of DLD-1-bearing NU/NU mice were plotted (dose: vvDD=1×10⁹ pfu; candidate VV deletion mutant=1×10⁶ pfu for all viruses). Statistical significance was calculated by log-rank test.

FIG. 26 shows VV tumour-efficacy. NU/NU mice were injected IP with A2780 tumour cells then treated with HBSS alone, vvDD, or a candidate VV deletion mutant (n=8) 12 days later and followed for survival. Kaplan-Meier curves of A2780-bearing NU/NU mice were plotted (dose: vvDD=1×10⁹ pfu, candidate VV deletion mutant=1×10⁶ pfu). Statistical significance was calculated by log-rank test.

FIG. 27 shows immune cells infiltration of VV-treated tumours. C57BL/6 and NU/NU mice were injected IP with MC38 or DLD1 tumour cells, respectively, and treated IP with HBSS, vvDD or candidate VVs 12 days later as follows: MC38-bearing C57BL/6 mice (vvDD=1×10⁹ pfu; ΔK1L VV=5×10⁷ pfu; ΔA46R VV=1×10⁷ pfu; ΔA52R VV=1×10⁷ pfu), and DLD-1-bearing NU/NU mice (all VVs: 5×10⁶ pfu). Mice were sacrificed 6 days post-treatment and tumours were harvested and fixed in formalin for analysis by immunohistochemistry. Quantification of immunohistochemistry staining for A) B220, B) CD3 (MC38-bearing C57BL/6 model only), C) F4/80, and D) Ly6G. Data depict percent of positively stained pixels relative to total number of pixels per tumour. E) Representative images of tumours stained for F4/80. Values are means of triplicates±SEM. § p<0.05 compared to mock-treated (HBSS), * p<0.05 compared to vvDD-treated.

### DETAILED DESCRIPTION OF THE DISCLOSURE

### I. Definitions

The term “vaccinia virus” or VV as used herein means any vaccinia virus strain including recombinant vaccinia strains. Vaccinia virus strains include Western Reserve, Lister, Wyeth, modified vaccinia Ankara (MVA), CV-1, NYCBH, Lederle, Temple of Heaven, Tashkent, USSR, Evans, Praha, LIVP, Ikeda, IHD, IHD-W, DPP25, IOC, Dls, LC16, EM-63, IC, Malbran, DUKE, Acambis, 3737, CVA, Copenhagen, Tian Tian and VJS6 strain/recombinant virus. For example, the recombinant viruses described below were made using parental Western Reserve (WR) vaccinia virus F13L+(wildtype virus with lacZ insertions (Roper & Moss, 1999)) parental viruses. Copenhagen strain, for example, or any strain comprising the gene to be modified could also be used as a parental strain to manufacture the VV comprising an inactivated poxvirus gene described herein.

The term “inactivated gene” refers to a gene comprising one or more mutations, such as a point mutation, a dominant negative mutation and/or a deletion mutation that produces a deletion of at least a portion of the gene, wherein either no protein is expressed or a biological function of the protein encoded by the gene (e.g. see Table 3), and/or a biological function of any complex in which the protein participates, is inactivated, e.g. reduced by at least 90%, at least 95%, or more and/or ablated, e.g. totally inhibited compared to a wild type molecule. The gene can be inactivated by recombinant methods, where for example the coding sequence or the gene can be deleted entirely and/or partially, or a mutation can be introduced ablating its biological function, for example enzymatic and/or structural functions of the encoded protein, the encoded protein can act as a dominant negative (such as a catalytic mutant) and form inactive complexes, and/or the encoded protein can be structurally and/or catalytically inactive (e.g. when the gene encodes an enzyme). Mutations can also be introduced by chemical selection. Preferably, the inactivation is a deletion mutant wherein all or a portion of the gene is deleted such that there is no expression of the protein normally encoded by the gene or at least no expression of a protein with biological function. For example portions of the gene that can be deleted include the start codon, a replicate of 1 or 2 nucleotides from the coding region such that the frame is disturbed or the entire coding portion of the gene. Also for example, the promoter of a gene can be deleted, inactivating the gene's normal function by inhibiting its expression.

As an example, “inactivated N1L gene” means a N1L gene comprising one or more mutations, such as a point mutation, a dominant negative mutation and/or a deletion mutation that produces a deletion of at least a portion of the gene wherein no protein is expressed or any encoded protein has no biological activity. The N1L coding sequence can be mutated, for example, by deleting or mutating sequence encoding one or more important binding residues, deleting a sequence encoding a BH3 binding domain or other mutation that inhibits N1L protein expression and/or biological activity. Similar definitions apply for the other poxvirus genes inactivated herein. Biological activities are provided in Table 3. A person skilled in the art, based on the present disclosure would readily, by comparing to wildtype and/or a mutant described herein, be able to determine if a particular mutation or deletion inactivated a poxvirus gene.

The term “left flanking portion of a poxvirus gene” refers to a nucleic acid cassette that includes sequence that is immediately upstream of the gene, part of the upstream portion of the gene (e.g. first exon) and does not include sequence relating to a different gene. For example depending on the orientation of the gene, the sequence can be immediately upstream of or include the 3′ part of the gene but does not include any upstream gene.

The term “right flanking portion of a poxvirus gene” refers to a nucleic acid cassette that includes sequence that is immediately downstream of the gene, part of the downstream portion of the gene (e.g. last exon) and does not include sequence relating to a different gene. For example, depending on the orientation of the gene, the sequence can be immediately downstream of or include the 5′ part of the gene but does not include any downstream gene.

The term “N1L” as used herein refers to the gene that encodes Protein N1 of viruses, including but not limited to poxviruses, such as vaccinia viruses. Alternative names for Protein N1 include, but are not limited to, “putative virulence factor”, “virokine”, and “N1L protein”. Also, viral N1L gene when referring to the vaccinia virus WR strain is denoted as VACWR028. The term “K1L” as used herein refers to the gene that encodes Interferon antagonist K1L of viruses, including but not limited to poxviruses, such as vaccinia viruses. Alternative names for Interferon antagonist K1L include, but are not limited to, “ankyrin-like protein”, “32.5 kDa protein”, and “Host range protein 1”. Also, viral K1L gene when referring to the vaccinia virus WR strain is denoted as VACWR032. The term “K3L” as used herein refers to the gene that encodes Protein K3 of viruses, including but not limited to poxviruses, such as vaccinia viruses. Alternative names for Protein K3 include, but are not limited to “interferon resistance protein” and “Protein K2”. Also, viral K3L gene when referring to the vaccinia virus WR strain is denoted as VACWR034. The term “A46R” as used herein refers to the gene that encodes Protein A46 of viruses, including but not limited to poxviruses, such as vaccinia viruses. Alternative name for Protein A46 includes, but is not limited to “Toll/IL1-receptor”. Also, viral A46R gene when referring to the vaccinia virus WR strain is denoted as VACWR172. The term “A52R” as used herein refers to the gene that encodes Protein A52 of viruses, including but not limited to poxviruses, such as vaccinia viruses. Alternative name for Protein A52 includes, but is not limited to “Toll/IL-receptor-like protein”. Also, viral A52R gene when referring to the vaccinia virus WR strain is denoted as VACWR178. A person skilled in the art would be familiar with the various nomenclatures used for poxvirus genes, such as vaccinia virus genes. For example, the more common, nomenclature for vaccinia genes uses letter-based designations (i.e. N1L and K1L).

The term “vvDD” or “double deleted VV” refers to a Western Reserve vaccinia virus with two non-essential gene deletions from its genome (i.e. deleted thymidine kinase (TK) and vaccinia growth factor (VGF); McCart et al., 2001).

The term “neoplastic disorder” as used herein refers to proliferative and/or dysplastic disorders including for example cancers of any kind and origin as well as precursor stages thereof, including for example, colon dysplasia and the like.

The term “cancer” as used herein refers to a cancer of any kind and origin including tumour-forming cells, blood cancers and/or transformed cells.

The term “neoplastic disorder cell” refers to one or more cells derived from or phenotypically similar to proliferative and/or dysplastic disorder cells such as cancer cells of any kind and origin as well as precursor stages thereof, including for example, neoplastic cells, precancer cells and/or tumour cells.

The term “cancer cell” includes cancer or tumour-forming cells, transformed cells or a cell that is susceptible to becoming a cancer or tumour-forming cell.

The term “a cell” includes a single cell as well as a plurality or population of cells administering a composition to a cell includes both in vitro and in vivo administrations.

The term “resistant cancer” or “chemotherapeutic resistant cancer” refers to a cancer that has decreased sensitivity to one or more chemotherapeutic drugs, for example by amplifying a gene that allows it to persist in the presence of the drugs. In an embodiment, a method of killing resistant cancer cells or chemotherapeutic resistant cancer cells comprises contacting the cancer cells or administering to a subject with a resistant cancer, a chemotherapeutic resistant cancer, or a tumour comprising resistant cancer cells or chemotherapeutic resistant cancer cells, an effective amount of a recombinant oncolytic poxvirus or viral DNA thereof described herein.

The term “peritoneal carcinomatosis” (PC) refers to a late stage cancer where cancer of primary tumour origin such as colon and ovary spreads to the peritoneum, disseminates throughout the peritoneal cavity.

The term “isolated poxvirus” as used herein includes but is not limited to naturally occurring, selected, such as chemically selected, and recombinant poxviruses that have been isolated, for example substantially purified, for example by a method known to a person of skill in the art. Methods such as plaque purification and limiting dilution can be used.

The term “recombinant poxvirus” refers to an engineered poxvirus, such as a vaccinia virus engineered to comprise a deletion that inactivates the activity of a gene product, that is generated in vitro using recombinant DNA technology and/or a poxvirus derived from such a recombined poxvirus (e.g., progeny virus).

The term “oncolytic” as used herein refers to a tumour-selective replicating virus that induces cell death in the infected cell, and/or tissue. Although normal or non-tumour cells may be infected, tumour cells are infected and lysed selectively in comparison to the normal or non-tumour cells. For example, an isolated poxvirus is oncolytic if it induces at least 2-fold, at least 5-fold, at least 6-fold, at least 10-fold, at least 15-fold, or at least 20-fold more cell death in a population of neoplastic disorder cells compared to control cells.

The term “cell death” as used herein includes all forms of cell death including for example cell lysis and/or apoptosis. Vaccinia virus for example is known to induce cell death by cell lysis and/or apoptosis. Cell death of a poxvirus infected cell and/or neighbouring cell may also refer for example to elimination of the cell by host immune system functions.

The term “normal tissue” as used herein refers to non-neoplastic tissue and/or tissue derived from a subject that is free of cancer of the particular tissue (e.g., when the tissue is pancreas, “normal tissue” can be derived from a subject that does not have pancreatic cancer). The term “normal cell of the same tissue type” as used herein refers to a cell or cells derived from such normal tissue.

As used herein, to “inhibit”, “reduce”, or “decrease” a function or activity, is any reduction in the function or activity when compared to otherwise same conditions except for a condition or parameter of interest, or alternatively, as compared to another condition.

As used herein “vector backbone” refers to a nucleic acid molecule that is used as a vehicle to deliver one or more nucleic acid molecules into a cell, e.g., to allow recombination. The vector backbone can refer optionally to the plasmid construct that is used to generate virus or to a virus genome (e.g., the non-recombined virus genome). Optionally, the vector backbone is constructed to permit deletion of one or more poxvirus genes (e.g., a shuttle plasmid created to facilitate homologous recombination with poxvirus to generate deletion mutants). A vector backbone into which has been inserted one or more nucleic acids to be transferred to a cell, is referred to as a vector construct, or a shuttle plasmid.

The term “isolated vector construct”, or “isolated recombinant vector” as used herein refers to a nucleic acid that is a vector construct substantially free of cellular material or culture medium when produced, for example, by recombinant DNA techniques.

The term “interrupter expression cassette” is used to refer to a polynucleotide that directs expression of one or more molecules that act as a cell marker and that optionally provides for a mode of isolating cells expressing said marker. The molecules are optionally used to select infected or transfected cells or to determine the efficiency of cell transduction or transfection. Molecules that are useful as cell markers, selection markers, or detection agents comprise, for example, fluorescent proteins such as EGFP, or derivatives thereof such as YFP and RFP, GFP or derivatives thereof such as YFP and RFP, enhanced GFP, mCherry, β-glucuronidase, β-galactosidase, HSA, xanthine-guanine phosphoribosyltransferase, luciferase enzymes such as firefly or renilla luciferase, etc. One skilled in the art will recognize that other fluorescent and non-fluorescent molecules can similarly be used.

The term “isolated nucleic acid” as used herein refers to a nucleic acid substantially free of cellular material or culture medium when produced by recombinant DNA techniques, or chemical precursors, or other chemicals when chemically synthesized. An “isolated nucleic acid” is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded. The nucleic acid sequences contemplated by the present disclosure include isolated nucleotide sequences which hybridize to a RNA product of a gene, nucleotide sequences which are complementary to a RNA product of a gene of the present disclosure, nucleotide sequences which act as probes, markers, or nucleotide sequences which are sets of specific primers.

The term “wildtype” is used to describe an object that can be found in nature as distinct from being artificially produced or manipulated by man. For example, a virus which can be isolated from a source in nature and which has not been intentionally modified by man in the laboratory, is naturally occurring.

The term “pfu” stands for plaque forming units and is a quantitative measure of live virus particles.

The term “primer” as used herein refers to a nucleic acid sequence, whether produced purified restriction digest or produced synthetically, which is capable of acting as a point of synthesis of when placed under conditions in which synthesis of a primer extension product, which is complementary to a nucleic acid strand is induced (e.g., in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer may contain 15-30 or more nucleotides, although it can contain less. The factors involved in determining the appropriate length of primer are readily known to one of ordinary skill in the art.

The term “sequence identity” as used herein refers to the percentage of sequence identity between two nucleic acid sequences or two polypeptide sequences. To determine the percent identity of two nucleic acid sequences or two amino acid sequences, the sequences are aligned for optimal comparison purposes (e.g., gaps can be introduced in the sequence of a first nucleic acid or amino acid sequence for optimal alignment with a second nucleic acid or amino acid sequence). The nucleotides or amino acid residues at corresponding nucleotide positions or amino acid positions are then compared. When a position in the first sequence is occupied by the same nucleotide or amino acid residue as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul (1990), modified as in Karlin and Altschul (1993). Such an algorithm is incorporated into the NBLAST and XBLAST programs of Altschul et al. (1990). BLAST nucleotide searches can be performed with the NBLAST nucleotide program parameters set, e.g., for score=100, wordlength=12 to obtain nucleotide sequences homologous to a nucleic acid molecules of the present disclosure. BLAST protein searches can be performed with the XBLAST program parameters set, e.g., to score=50, wordlength=3 to obtain amino acid sequences homologous to a protein molecule of the present disclosure. To obtain gapped alignments for comparison purposes, Gapped BLAST can be utilized as described in Altschul et al. (1997). Alternatively, PSI-BLAST can be used to perform an iterated search which detects distant relationships between molecules (Altschul et al., 1997). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller (1988). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “treating” or “treatment” as used herein is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage neoplastic disorder can be treated to prevent progression or alternatively a subject in remission can be treated with an isolated or recombinant poxvirus or composition described herein to prevent recurrence. Treatment methods comprise administering to a subject a therapeutically effective amount of one or more isolated or recombinant poxvirus or compositions described in the present disclosure, and optionally consist of a single administration, or alternatively comprise a series of applications. For example, the isolated and/or recombinant poxviruses and compositions described herein may be administered at least once a week, from about one time per week to about once daily for a given treatment or the isolated, or recombinant poxviruses and/or compositions described herein may be administered twice daily. As another example, the isolated or recombinant poxvirus is administered once only, or for example every 3 weeks for 4 cycles. The length of the treatment period depends on a variety of factors, such as the severity of the disease, the age of the patient, the concentration, the activity of the isolated or recombinant poxviruses and/or compositions described herein, and/or a combination thereof. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

The dosage administered will vary depending on the use and known factors such as the pharmacodynamics characteristics of the particular biologic, such as a recombinant poxvirus, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

The terms “reducing,” or “preventing” or any variation of these terms, when used in the claims and/or the specification in the context of a treatment includes any measurable decrease or complete inhibition to achieve a desired result. Desired results include but are not limited to palliation, reduction, slowing, or eradication of a cancerous or proliferative or dysplastic condition, as well as an improved quality or extension of life.

The term “potency” as used herein refers to a measure of viral activity expressed in terms of the amount for example in pfu, required to produce an effect of given intensity. For example, higher potency virus evokes a given response at lower concentrations, while lower potency virus may evoke the same response at higher concentrations.

The term “subject” as used herein includes all members of the animal kingdom including mammals, and suitably refers to humans.

As used herein, “contemporaneous administration” and “administered contemporaneously” means that two biologics, such as two recombinant poxviruses, are administered to a subject such that they are both biologically active in the subject at the same time. The exact details of the administration will depend on the pharmacokinetics of the two biologics in the presence of each other, and can include administering one substance within 24 hours of administration of the other, if the pharmacokinetics is suitable. Designs of suitable dosing regimens are routine for one skilled in the art. In particular embodiments, two biologics will be administered substantially simultaneously, i.e., within minutes of each other, or in a single composition that comprises both substances.

As used herein, the phrase “effective amount” or “therapeutically effective amount” means an amount effective, at dosages and for periods of time necessary to achieve the desired result. For example in the context or treating a neoplastic disorder, an effective amount is an amount that for example induces remission, reduces tumour burden, and/or prevents tumour spread or growth compared to the response obtained without administration of the isolated or recombinant poxviruses and/or compositions described herein. Effective amounts may vary according to factors such as the disease state, age, sex, weight of the subject. The amount of a given isolated or recombinant poxvirus and/or composition described herein that will correspond to such an amount will vary depending upon various factors, such as the given isolated or recombinant poxvirus and/or composition described herein, the pharmaceutical formulation, the route of administration, the type of disease or disorder, the identity of the subject or host being treated, and the like, but can nevertheless be routinely determined by one skilled in the art.

The use or administration of composition comprising recombinant poxviruses to a subject can comprise injection. The route of injection includes but not limited to intradermal, subcutaneous, intramuscular, intravenous, intraosseous, intraperitoneal, intrathecal, intracerebral, and intratumoural, such as described in U.S. Pat. Nos. 5,399,363, 5,543,158, and 5,641,515, each incorporated herein by reference in its entirety.

Intratumoural injection or injection directly into the tumour vasculature is specifically contemplated for discrete, solid, accessible tumours. Local, regional or systemic administration also may be appropriate. For tumours of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumour, spaced at approximately 1 cm intervals. In the case of surgical intervention, the present invention may be used preoperatively, to render an inoperable tumor subject to resection. Continuous administration also may be applied where appropriate, for example, by implanting a catheter into a tumor or into tumor vasculature. Such continuous perfusion may take place for a period from about 1-2 hours, to about 2-6 hours, to about 6-12 hours, or about 12-24 hours following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

In certain embodiments, the tumour being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumour site.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this specification and the appended claims, the singular forms “a”, “an” and “the” include plural references unless the content clearly dictates otherwise. Thus, for example, a composition containing “a virus” includes a mixture of two or more viruses. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art

### II. Methods, Uses, Viruses, Vectors, and Compositions

The disclosure relates to recombinant oncolytic poxviruses, for example vaccinia virus, with inactivated poxvirus genes, for example N1L, K1L, K3L, A46R and/or A52R, recombinant vectors for making said recombinant oncolytic poxviruses and methods of using these viruses. These recombinant oncolytic poxviruses exhibit an enhanced potency in that they evoke similar anti-tumour efficacy at lower concentration as compared to vvDD, while maintaining tumour-selectivity.

An aspect includes a recombinant oncolytic poxvirus, such as vaccinia virus (VV), or viral DNA thereof, comprising one or more inactivated poxvirus genes selected from N1L, K1L, K3L, A46R, and/or A52R, preferably selected from K1L, A46R and/or A52R.

In an embodiment, the one or more inactivated poxvirus genes are inactivated by deletion of all or a portion of the gene. In one embodiment, the entire gene is deleted from the recombinant poxvirus. In another embodiment, a portion of the gene, such as the start codon, is mutated or deleted from the recombinant poxvirus. In another embodiment, the reading frame of the gene is changed such that the gene would not be expressed.

In an embodiment, the inactivated VV genes are inactivated by replacing the poxvirus gene with a detectable interrupter expression cassette, optionally the detectable interrupter expression cassette comprising one or more of the components selected from a fluorescent protein, a luciferase enzyme and a gpt protein, and wherein the poxvirus gene is selected from N1L, K1L, K3L, A46R and A52R, preferably K1L, A46R and A52R.

In other embodiments, the inactivated VV genes are inactivated by replacing the poxvirus gene with an inactivated version of the poxvirus gene, for example deleted for the start codon, a required domain etc such that the replaced inactivated version either does not produce any expression product or produces only an inactive expression product with inactivated biological function (e.g. no or virtually no activity (e.g. less than 10%) of an activity for the activities listed in Table 3).

In an embodiment, the recombinant oncolytic poxvirus or viral DNA thereof is constructed using a recombinant vector described herein.

In an embodiment, the recombinant oncolytic poxvirus or viral DNA thereof comprises two, three, four or five inactivated genes selected from N1L, K1L, K3L, A46R, A52R, TK and growth factor genes such as VGF.

In an embodiment, the recombinant oncolytic poxvirus or viral DNA thereof further comprises an inactivated TK gene and one or more inactivated growth factor genes, optionally VGF.

In some embodiments, the recombinant oncolytic poxvirus or viral DNA thereof is isolated.

The group of poxviruses that are expected to be useful include for example poxviruses that are able to infect mammalian cells, particularly human cells and which in their wild type form express immunomodulatory genes N1L, K1L, K3L, A46R and/or A52R. Accordingly in an embodiment, the wild type poxvirus comprises immunomodulatory genes N1L, K1L, K3L, A46R and/or A52R and is infectious for mammalian cells. In an embodiment, the poxvirus is infectious for human cells. In a further embodiment, the poxvirus is optionally an Orthopoxvirus such as a vaccinia virus, a Leporipoxvirus, a Suipoxvirus, a Capripoxvirus, a Cervidpoxvirus, an Avipoxyiurs, a Molluscipoxvirus, a Parapoxvirus, a Yatapoxvirus, a Myoxa virus, an Orf virus and a Crocodylidpoxvirus. Vaccinia viruses for example are useful as oncolytic agents. Vaccinia viruses, as well as many other Orthopoxviruses (e.g. ECTV), have a quick and efficient life cycle, forming mature virions in the order of 6 h and vaccinia virus spreads efficiently cell to cell thus increasing the efficacy of an in vivo infection. Vaccinia viruses can infect a wide range of human tissues and there is a large body of knowledge about its biology and extensive experience with it clinically as part of the smallpox vaccination program. Accordingly, in a preferred embodiment, the poxvirus is a vaccinia virus.

The experiments disclosed herein have been conducted in a laboratory adapted strain of vaccinia virus. A number of laboratories adapted and clinical strains are known to a person of skill in the art. For human applications, a clinical grade virus is useful. Accordingly in one embodiment, the isolated and/or recombinant poxvirus is a clinical grade virus. In an embodiment, the vaccinia virus is a vaccinia virus strain selected from Western Reserve (WR; Genbank accession: NC_006998.1), Tian Tian (AF095689), NYCBH, Wyeth, Copenhagen (M35027), Lister (AY678276), modified vaccinia Ankara (MVA; U94848), Lederle, Temple of Heaven, Tashkent, USSR, Evans, Praha, LIVP, Ikeda, IHD, IHD-W, DPP25, IOC, Dls, LC16 (AY678275), EM-63, IC, Malbran, DUKE (DQ439815), Acambis (AY313847), 3737 (DQ377945), CVA (AM501482), VJS6 and AS each of the foregoing incorporated herein by reference. In a preferred embodiment, the strain is Western Reserve or Copenhagen.

Another aspect includes a recombinant vector comprising a left flanking portion of a poxvirus gene selected from N1L, K1L, K3L, A46R, and A52R gene and a right flanking portion of said poxvirus gene, wherein the left flanking portion and the right flanking portion are operably linked to a detectable interrupter expression cassette, optionally comprising one or more of the components selected from a fluorescent protein, a luciferase enzyme and a gpt protein, and wherein the poxvirus gene is selected from N1L, K1L, K3L, A46R and A52R, preferably K1L, A46R and A52R. In an embodiment, the detectable interrupter expression cassette directs the expression of one or more polypeptides, optionally a fluorescent protein, a luciferase enzyme or a gpt protein.

In an embodiment, the recombinant vector is a shuttle plasmid, optionally comprising one or more of the components selected from a fluorescent protein, a luciferase enzyme and a gpt protein, and wherein the poxvirus gene is selected from N1L, K1L, K3L, A46R and A52R, preferably K1L, A46R and A52R.

In an embodiment, each flanking portion of a poxvirus gene comprises between 150 and 700 nucleotide residues, such as at least 150, 200, 250, 300, or 350 nucleotide residues, and at most 400, 450, 500, 550, 600, 650 and 700 nucleotide residues. In an embodiment, each flanking portion comprise about 200-600 nucleotide residues, preferably 300-500 nucleotide residues, of flanking sequence.

In an embodiment, the flanking sequence has at least 80%, at least 85%, at least 90%, at least 95%, at least 97%, at least 98%, at least 99% or has 100% sequence identity to corresponding sequence in Accession number: NC_006998.1 or is for example from VJS6 virus. In an embodiment, the flanking sequence has at least 80%, at least 85%, at least 90%, at least 95%, at least 97%, at least 98%, at least 99% or has 100% sequence identity to corresponding sequence in a vaccinia virus strain selected from Tian Tian (AF095689), NYCBH, Wyeth, Copenhagen (M35027), Lister (AY678276), modified vaccinia Ankara (MVA; U94848), Lederle, Temple of Heaven, Tashkent, USSR, Evans, Praha, LIVP, Ikeda, IHD, IHD-W, DPP25, IOC, Dls, LC16 (AY678275), EM-63, IC, Malbran, DUKE (DQ439815), Acambis (AY313847), 3737 (DQ377945), CVA (AM501482) and AS. Each of the foregoing strain is incorporated herein by reference.

In an embodiment, the isolated or recombinant vaccinia virus WR strain comprises an inactivated N1L, K1L, K3L, A46R, and/or A52R which is deleted for at least 2, at least 5, at least 10, at least 20, at least 30, at least 35, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 350, at least 400, at least 500, at least 750, at least 1000, at least 2000, at least 3000 nucleotides residues of Accession number: NC_006998.1. In another embodiment, the isolated or recombinant poxvirus and/or vaccinia virus comprises an inactivated N1L, K1L, K3L, A46R, and/or A52R by insertion, deletion and/or mutation for at least 1, at least 2, at least 5, at least 10, at least 20, at least 30, at least 35, at least 40, at least 50, at least 60, at least 61, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 350, at least 400, at least 500, at least 750, at least 1000, at least 2000, at least 3000 nucleotide residues, wherein the vaccinia virus is at least 80%, at least 85%, at least 90%, at least 95, at least 98%, at least 99% or more identical to nucleotide residues of Accession number: NC_006998.1. The insertion, deletion and/or mutation can also be described referring to specific genomic positions for a particular strain, e.g. WR strain. Accordingly in an embodiment, nucleotides corresponding to nucleotides 21819-22172, 25071-25925, 27306-27572, 153147-153869 and/or 158743-159315 of WR genome are deleted. In an embodiment, the inactivated N1L gene comprises a disruption in the N1L ORF such that an insertion, a deletion or a mutation is made in between nucleotides 21819 and 21821 in the WR genome which causes disruption of the start codon. In another embodiment, the inactivated K1L gene comprises a disruption in the K1L ORF such that an insertion, a deletion or a mutation is made in between nucleotides 25071 and 25073 in the WR genome which causes disruption of the start codon. In another embodiment, the inactivated K3L gene comprises a disruption in the K3L ORF such that an insertion, a deletion or a mutation is made in between nucleotides 27306 and 27308 in the WR genome which causes disruption of the start codon. In another embodiment, the inactivated A46R gene comprises a disruption in the A46R ORF such that an insertion, a deletion or a mutation is made in between nucleotides 153147 and 153149 in the WR genome which causes disruption of the start codon. In yet another embodiment, the inactivated A52R gene comprises a disruption in the A52R ORF such that an insertion, a deletion or a mutation is made in between nucleotides 158743 and 158745 in the WR genome which causes disruption of the start codon. A person skilled in the art would readily be able to determine the corresponding positions in other strains. In an embodiment, the recombinant oncolytic poxvirus or viral DNA thereof is made according to a method described herein.

The deletion can also be described in terms of amino acid positions. For example, a deletion of at least 90 nucleotides of N1L, K1L, K3L, A46R, and/or A52R corresponds to a deletion of at least 30 amino acid residues. A further aspect includes a cell transfected with the recombinant vector described herein, and optionally infected with wildtype WR VV.

It is also disclosed herein that additional functional inactivation, e.g. gene deletion or mutation, of other poxvirus genes such as thymidine kinase (also referred to as TK or J2R) and/or VGF, can be combined with the N1L, K1L, K3L, A46R and/or A52R. Accordingly in an embodiment, the virus further comprises an inactivated TK and/or VGF gene. Mutations including point mutations, dominant negative mutations and deletions that affect activity or expression levels are useful with the present methods. In an embodiment, the inactivated TK gene comprises a disruption in the TK ORF such that an insertion, a deletion or a mutation is made in between nucleotides 81001 and 81002 in the WR genome which causes disruption between amino acid 92 and 93 such that only the first 92 residues of TK are expressed. In another embodiment, the inactivated VGF gene comprises a disruption in the VGF ORF such that an insertion is made in between nucleotides 7333 and 7335, and/or between nucleotides 186957 and 186959 in the WR genome which causes disruption of the start codon.

Another aspect includes a cell infected by the recombinant oncolytic poxvirus, such as vaccinia virus, and/or comprising poxvirus DNA described herein.

In an embodiment, the cell is a neoplastic disorder cell optionally a cancer cell, optionally a solid tumour cell or a blood cancer cell. The cell can be any tumour or cancer cell. In an embodiment, the solid tumour cell is optionally an ovarian cancer cell, a colon cancer cell, a hepatocellular carcinoma cell, a lung cancer cell, a mesothelioma cell, a prostate cancer cell, a melanoma cell, a renal cell carcinoma cell, a head and neck cancer cell, a pancreatic cancer cell, a glioma cell, a gastric cancer cell, and a breast cancer cell. In an embodiment, the blood cancer is myeloma or leukemia. In an embodiment, the cancer cell is a lymphoma cell. The cell can be any cell including for example a cell type mentioned herein, and the skilled person can readily identify cells suitable for recombinant oncolytic poxvirus infection.

A further aspect is a composition comprising the recombinant vector, cell or recombinant oncolytic poxvirus or viral DNA thereof described herein and a suitable diluent optionally a pharmaceutically suitable diluent.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of pfu for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles, including all values and ranges there between, to the tumour or tumour site. In an embodiment, the composition comprises recombinant oncolytic poxvirus, wherein the concentration of the recombinant oncolytic poxvirus is between 1×10⁴ and 1×10¹⁵ pfu/dose, such as at least about 1, 10, 1000, 1×10⁴, 1×10⁵, 1×10⁶, 2×10⁶, 3×10⁶, 4×10⁶, 5×10⁶, 6×10⁶, 7×10⁶, 8×10⁶, 9×10⁶, 1×10⁷, or 2×10⁷ pfu/dose, and at most 5×10⁶, 6×10⁶, 7×10⁶, 8×10⁶, 9×10⁶, 1×10⁷, 2×10⁷, 3×10⁷, 4×10⁷, 5×10⁷, 1×10⁸, 1×10⁹, 1×10¹⁰, 1×10¹¹, 1×10¹², 1×10¹³, 1×10¹⁴, or 1×10¹⁵ pfu/dose. In another embodiment, concentration of the recombinant oncolytic poxvirus is between 1×10⁶ and 5×10⁷ pfu/dose, optionally 1×10⁶, 1×10⁷, or 5×10⁷ pfu/dose.

Also provided in another aspect is an in vitro method of making the recombinant oncolytic poxvirus, or viral DNA thereof described herein comprising introducing the recombinant vector described herein into cells infected with a poxvirus, under conditions suitable for recombination, and isolating the recombinant oncolytic poxvirus or viral DNA thereof comprising inactivated poxvirus gene selected from N1L, K1L, K3L, A46R, and A52R.

Another aspect is a method of killing neoplastic disorder cells, optionally cancer cells, the method comprising contacting the neoplastic disorder cells, optionally cancer cells or administering to a subject with a neoplastic disorder such as a cancer or tumour comprising cancer cells, an effective amount of the recombinant oncolytic poxvirus or viral DNA described herein. In an embodiment, the method is for reducing tumour burden and/or treating a subject afflicted with a solid tumour comprising solid tumour cells. In an embodiment, the subject has metastatic disease and/or late stage cancer and optionally has peritoneal carcinomatosis (PC). In another embodiment, the method comprises administrating the recombinant oncolytic virus prior to, concurrently, or after chemotherapy, palliative surgery, cytoreductive surgery, and/or hyperthermic intraperitoneal chemotherapy, to a cancer patient in need.

Uses and compositions for use for killing cancer cells, reducing tumour burden and treating a subject afflicted with a solid tumour are also provided.

In an embodiment, the recombinant oncolytic poxvirus or composition is administered to a subject by injection. In another embodiment, the route of injection includes but not limited to intradermal, subcutaneous, intramuscular, intravenous, intraosseous, intraperitoneal, intrathecal, intracerebral, and intratumoural.

The following non-limiting Examples are illustrative of the present disclosure:

### EXAMPLES

### Example 1

### Cell Lines

Human colorectal adenocarcinoma cell line DLD-1, human ovarian cancer cell line A2780, and normal monkey kidney fibroblast cell line CV-1 were obtained from the American Type Culture Collection (ATCC; Manassas, Va., USA). MC38 murine colorectal adenocarcinoma cell line cells and C57BL/6 murine sarcoma cell line 24JK were obtained from the National Institute of Health (NIH; Bethseda, Md., USA). Cells were cultured at 37° C. and 5% CO₂ in media supplemented with 10% fetal bovine serum (FBS; PAA Laboratories, Etobicoke, ON, Canada) and 1% penicillin-streptomycin (Invitrogen, GIBCO, Grand Island, N.Y., USA). All cell lines were maintained in Dulbecco's Modified Eagle Medium (DMEM; Sigma-Aldrich, St. Louis, Mont., USA) except A2780 cells which were maintained in Roswell Park Memorial Institute medium (RPMI 1640; in-house Toronto Medical Discovery Tower media, University Health Network, Toronto, ON, Canada).

### Vaccinia Viruses

Western Reserve (WR) vaccinia virus F13L+(wildtype virus with lacZ insertions (Roper & Moss, 1999)) was used as the backbone virus in the creation of candidate viruses. All candidate viruses were compared to the previously described virus, vvDD (McCart et al., 2001), a WR vaccinia virus with a deletion in its thymidine kinase and vaccinia growth factor genes. Specifically, vvDD-R2R-Luc is used. The viral TK gene is interrupted with a sequence for red fluorescent protein (RFP) insertion conjoined with Renilla luciferase via a foot-and-mouth disease virus 2A motif to allow for bicistronic marker expression and the VGF genes found within the VV inverted terminal repeats are interrupted by a lacZ gene at both ends of the virus genome (Lun et al., 2009).

### Creation of the Candidate Vaccinia Virus Deletion Mutants

Wildtype WR vaccinia virus, F13L+, was used as a backbone virus for the creation of the vaccinia virus deletion mutants via homologous recombination by using a vaccinia virus shuttle plasmid. The final candidate viruses were wildtype WR vaccinia viruses with a deletion in one of the following genes: N1L, K1L, K3L, A46R, or A52R. The shuttle plasmid, pVX-R2R-Luc, has a RFP gene fused to Renilla luciferase via a foot-and-mouth disease virus 2A motif to facilitate bicistronic marker expression under the control of a synthetic late promoter pSyn/late and a xanthine-guanine phosphoribosyltransferase (gpt or alternately abbreviated xgprt) gene under regulation of the early/late p7.5 promoter. Five hundred base pair gene segments flanking the right and left regions of the wildtype candidate genes for deletion were generated by polymerase chain reaction (PCR) with the VJS6 virus DNA (VGF-WR vaccinia virus with lacZ gene insertions). The left flanking gene segments were digested with MfeI and SpeI and ligated between the MfeI and NheI (which has compatible ends with an SpeI digestion) on the plasmid. The right flanking gene segments were digested and ligated between the BssHII and PvuII sites on the plasmid. The final shuttle plasmids had the RFP/Renilla luciferase fusion and xgprt genes and the promoters mentioned above flanked with wildtype gene segments from the candidate genes to allow for homologous recombination. Monolayers of CV-1 cells at 80-90% confluence were infected with 1.25×10⁴ F13L+(Western Reserve strain vaccinia virus with a lacZ insertion) in 500 μl of low-serum media (2.5% FBS) at 37° C. for 2 hours with intermittent shaking every 15 minutes. The supernatant was removed and a liposomal transfection (Lipofectamine 2000; Invitrogen, GIBCO) was conducted with OptiMEM media (Invitrogen, GIBCO) containing 2.5 μg shuttle plasmid DNA and 10 μl liposomes/well at 37° C. for 4 hours. The transfection media was removed and replaced with drugged media (10% FBS, supplemented with 250 μg/ml xanthine, 14.9 μg/ml hypoxanthine, 25 μg/ml mycophenolic acid; Sigma Aldrich) for selection. After 3 days of incubation at 37° C., cells were collected in drugged low-serum media (2.5% FBS), subjected to 3 freeze-thaw cycles, sonicated, and serially diluted (1/4, 1/5, and 1/10). Each dilution was re-infected in duplicate in 500 μl drugged low serum media (2.5% FBS) at 37° C. into a monolayer of confluent CV-1 cells that were incubated with drugged media for at least 24 hours. After 2 hours, the supernatant was replaced with 2 ml agarose/drugged media overlay and incubated at 37° C. Three days later, the monolayers were observed for RFP expression and viral plaque formation. Six RFP-positive plaques were isolated and resuspended in drugged low-serum media (2.5% FBS, supplemented with 250 g/ml xanthine, 14.9 μg/ml hypoxanthine, 25 μg/ml mycophenolic acid) for re-infection into another monolayer of drugged CV-1 cells. After 5-6 rounds of selection, all plaques were positive for RFP for all candidate viruses.

TABLE 1
Primers for producing inserts
for the shuttle plasmid

| Poxvirus | | Left | Right |
|---|---|---|---|
| Genes | | Flanking Insert | Flanking Insert |
| N1L | Fwd | gatccaattgcctaa | gatcgcgcgcgtaca |
| | | ctctttcgaatactt | tacatcgccgtcatc |
| | | (SEQ ID NO: 1) | (SEQ ID NO: 3) |
| | Rev | gatcgctagcggaag | gatccagctgttatg |
| | | agtcattcaccatac | gaggatatgtgaacg |
| | | (SEQ ID NO: 2) | c |
| | | | (SEQ ID NO: 4) |
| K1L | Fwd | gatccaattgtgacg | gatcgcgcgotttgc |
| | | tacatgagtctgagt | atgttaccactatca |
| | | (SEQ ID NO: 5) | (SEQ ID NO: 7) |
| | Rev | gatcgctagccgtgg | gatccagctgcagac |
| | | atatgatgattctct | atggatctgtcacga |
| | | (SEQ ID NO: 6) | (SEQ ID NO: 8) |
| K3L | Fwd | gatccaattgtaccg | gatcgcgcgcataat |
| | | gatctacgttctact | ccttctcgtatac |
| | | (SEQ ID NO: 9) | (SEQ ID NO: 11) |
| | Rev | gatcgctagcggata | gatccagctgtgctg |
| | | tatagatgtcaatta | atcctcccattccgt |
| | | (SEQ ID NO: 10) | (SEQ ID NO: 12) |
| A46R | Fwd | gatccaattgcacga | gatcgcgcgctgact |
| | | taatatcagaggagt | tacttgtataataag |
| | | (SEQ ID NO: 13) | (SEQ ID NO: 15) |
| | Rev | gatcgctagccttca | gatccagctgcagaa |
| | | ttacgtatgactaat | catgtagacgaatca |
| | | (SEQ ID NO: 14) | (SEQ ID NO: 16) |
| A52R | Fwd | gatccaattgcggga | gatccccgggacgcg |
| | | gacgaggatatagct | tgacaatgatgcgga |
| | | (SEQ ID NO: 17) | agaaca |
| | | | (SEQ ID NO: 19) |
| | Rev | gatccccgggaggcc | gatcgacttgagcgt |
| | | tatagacctagtaca | catctggtagataga |
| | | taaaa | ccatcg |
| | | (SEQ ID NO: 18) | (SEQ ID NO: 20) |

Abbreviations:
Fwd, forward;
Rev, reverse.

### Viral DNA Extraction and PCR

Confluent CV-1 monolayers were infected with half of each isolated plaque from the 5^{th} or 6^{th} round of selection or a small amount from the CV-1 propagation during virus production until complete cytopathic effect was seen. Cells were washed and collected in 1 ml homemade PCR buffer [50 mM KCl (Sigma Aldrich), 10 mM Tris HCl, pH 8 (Fisher Scientific, Fair Lawn, N.J., USA), 2.5 mM MgCl₂, 0.1 mg/ml gelatin, 0.45% Tween 80, and 0.45% NP40 (BioShop, Burlington, Ontario, Canada)]. Viral DNA was released from cells by Proteinase K treatment (ThermoScientific, Lithuania) at 55° C. for an hour, then at 95° C. for 10 minutes to deactivate the enzyme prior to PCR. A standard PCR with the crude viral DNA extract was conducted with the primers originally used to produce inserts for the shuttle plasmid. Specifically, the forward primer from the left flanking insert and the reverse primer from the right flanking insert were used (Table 1). PCR validation was conducted after the 5^{th}-6^{th} cycle of selection, after propagation in CV-1 cells during virus stock production and at the end of the virus stock production (See Virus Stock Production below).

### Virus Stock Production

Half a plaque from the 5^{th}-6^{th} cycle of virus selection was reinfected into a monolayer of CV-1 cells in a 6-well plate. When complete cytopathic effect was seen, the cells were harvested with a CellScraper (Sarstedt, Sarstedt AG & Co, Germany) and reinfected into confluent CV-1 monolayers in two 175 cm² flasks for 2 days and collected. After PCR re-validation with a small amount of cell suspension (see above), the candidate virus was propagated by reinfecting into thirty 144 cm² plates of confluent 24JK cells. After 3 days, infected 24JK cells were harvested and centrifuged at 1500 rpm for 10 minutes (SLA-1500 rotor, Sorvall; ThermoScientific). The cell pellet was resuspended in 20 ml Tris-Cl (pH 9.0), subjected to 3 freeze/thaw cycles and mechanically homogenized with a sterile Dounce Grinder (Sigma-Aldrich). Ultracentrifugation of the cell lysate was performed at 25,000 rpm for 80 minutes (XL-70 Ultracentrifuge; Beckman Coulter, Brea, Calif., USA) at 4° C. over a 36% sucrose cushion. Finally, the virus pellet was resuspended in Tris-Cl (pH 9.0), aliquoted, and stored at −80° C.

### Virus Plaque Assay

CV-1 cells were seeded in 6-well plates with 3×10⁵ cells/well and incubated at 37° C. for 2 days. Infected samples were subjected 3 freeze/thaw cycles and sonicated to release the virus from the cells. Viral lysates were serially diluted in low serum media (2.5% FBS) and used to infect the CV-1 monolayers with 400 μl per well at 37° C. with intermittent shaking every 10 minutes. Two hours later, 1.5 ml of media (10% FBS) was added and incubated at 37° C. Two days later, the cell monolayers were stained with crystal violet and plaques were counted.

### Viral Replication

Tumour cells (DLD-1, A2780) were seeded in 6-well plates until confluent, at which point the number of cells were determined. MC38 were seeded in 6-well plates with 5×10⁶ cells/well and incubated overnight to achieve 80-90% confluency. Cells were infected at an MOI of 0.1 of either vvDD or one of the candidate viruses in 0.5 ml low serum media (2.5% FBS) at 37° C. for 2 hours with intermittent shaking every 10-15 minutes. Cells were supplemented with media (10% FBS) and incubated at 37° C. Cells and supernatants were harvested in triplicates at each time point (2, 24, 48, and 72 hpi using a Cell Scraper (Sarstedt, Sarstedt AG & Co, Germany) and stored at −80° C. until use. Virus was quantified by viral plaque assays on CV-1 cells where CV-1 cells were seeded at 3×10⁵ cells/well in 6-well plates and incubated for 2 days at 37° C. until confluency. The virus treated samples were subjected to 3 freeze/thaw cycles and sonicated on ice before serial dilution in DMEM-2.5% FBS. Subsequently, 0.4 ml of the dilutions were added into a confluent well of CV-1 cells and incubated at 37° C. for 2 hours with intermittent shaking and then supplemented with DMEM-10% FBS and incubated at 37° C. Two days later, the monolayers were stained with crystal violet and plaques were counted.

### Viral Cytotoxicity

MC38 cells were seeded in a 96-well plate with 5×10³ cells/well and incubated overnight at 37° C. before infection. A2780 and DLD-1 cells were seeded in 96-well plates with 1×10⁴ cells/well and incubated overnight at 37° C. before infection. Cells were infected at an MOI of 0.1, 1, or 5 in triplicate with either vvDD or one of the candidate viruses in 25 μl of media (2.5% FBS) for 2 hours at 37° C., then supplemented with 75 μl media (10% FBS). Cytotoxicity was evaluated at 2, 24, 48, and 72 hpi with 3-(4,5-dimethyl-2-yl)-5-(3-carboxymethoxyphenyl)-2-(4-sulfophenyl)-2H-tetrazolium (MTS) cell viability assay (CellTiter96® Aqueous One Solution, Promega, Madison, Wis., USA) according to the manufacturer's protocol. Relative cell viability was calculated by dividing the absorbance of infected wells with the absorbance of the mock-infected wells.

### Measuring Viral Spread by Red Fluorescent Protein (RFP) Expression

Experiments were conducted as described in the viral replication protocol. Before harvesting the cell monolayers, fluorescence microscopy was conducted to acquire brightfield and RFP images (acquired under the Cy3 lens) at 10× magnification using the Zeiss AxioObserver microscope (Carl Zeiss, Oberkochen, Germany) equipped with a Series 120Q Fluorescence Illumination unit (EXFO, Quebec City, Quebec, Canada). Images were taken with a Zyla 5.5 sCMOS camera (Andor Technologies, Belfast, United Kingdom). Percent area infected was measured with the Fiji ImageJ software with a uniform threshold based on RFP presence within the image.

### Tumour Spheroid Generation, Infection, and Analysis

Spheroids were cultured using tumour cell lines MC38 and DLD-1. Cells were dissociated with Accutase and seeded at 1,000 cells/well in 96-well round-bottomed plates coated with 1% polyHEMA (Sigma Aldrich). Plates were subsequently spun at 1,500 rpm for 10 minutes and incubated at 37° C. for 72 hours prior to infection. vvDD, one of the candidate VVs (MOI=2) or media alone DMEM-2.5% FBS was used to infect the spheroids in triplicate for 2 hours at 37° C. and supplemented with media DMEM-10% FBS. Infection was confirmed by RFP expression visualized with a LSM 700 Confocal Microscope (Carl Zeiss). Tumour spheroid size was measured with the Fiji ImageJ software. Briefly, brightfield images of the spheroids were converted to 8-bit greyscale images where spheroids were converted into a binary mask and the major and minor axis of a fit elliptical were measured. Tumour volume was calculated based on the following equation assuming spheroid shape: V=4/3*π ([major axis]/2)([minor axis]/2)². Clonogenic assays were also conducted 96 hpi. Approximately 30-50 spheroids were collected, centrifuged, and disaggregated with trypsin. Live cells were plated in 6-well plates at 25-100 cells/well in duplicate and incubated for 10 days before crystal violet staining. Surviving fraction was calculated by the number of colonies (>50 cells) divided by the number of cells originally seeded.

### Mice

Female C57BL/6 mice and NU/NU athymic nude mice (Jackson Laboratory, Bar Harbor, Me., USA) were used as syngeneic and xenograft models, respectively. All mice were housed under standard conditions and given food and water ad lib. Experimental protocols were approved by the Animal Care Centre, University Health Network, Toronto. Mice were euthanized by CO₂ asphyxiation when signs of morbidity were present including: viral toxicity, a subcutaneous tumour at the injection site >1.5 cm, difficulty breathing, moribund, cachectic or inability to obtain food or water.

### In Vivo Toxicity Studies

Female C57BL/6 mice or NU/NU athmymic nude mice (n=6) were injected with the indicated doses of virus in HBSS+0.1% bovine serum albumin (BSA) or HBSS+0.1% BSA alone and observed for survival. Body weight was measured every 2-3 days.

### Syngeneic Model

Female C57BL/6 mice were injected intraperitoneally (IP) with 10⁵ MC38 cells in serum-free media. Twelve days post-tumour injection, mice were injected with the indicated doses of virus in 1 ml Hanks-Buffered Saline Solution (HBSS; Invitrogen, GIBCO)+0.1% BSA or HBSS+0.1% BSA alone. Mice were followed for survival (n=8) or sacrificed 6 days post-infection for biodistribution studies (n=3).

### Xenograft Model

Female NU/NU athymic nude mice were injected IP with either 5×10⁶ DLD-1 cells or 10⁷ A2780 cells in serum-free media. Twelve days post-tumour injection, mice were injected with the indicated doses of virus in HBSS+0.1% BSA or HBSS+0.1% BSA alone. Mice were followed for tumour survival (n=8) or sacrificed 6 days post-infection for biodistribution studies (n=3).

### Biodistribution

Mice from the syngeneic or xenograft model had tumours and virus injected as described above and then euthanized 6 days post-infection and the tumours, ovaries, kidneys, bowel, liver, spleen, lung, heart, brain and bone marrow were harvested and stored at −80° C. in HBSS. Tissues were homogenized with the TissueLyzer II (Qiagen, Hilden, Germany), subject to 3 freeze-thaw cycles, and sonicated prior to the viral plaque assay with CV-1 cells. Titers were normalized to total protein per sample (mg) measured with the Pierce™ bicinchoninic acid (BCA) protein assay kit (Thermo Fisher Scientific, Waltham, Md., USA). Relative titers in normal tissues were calculated by dividing the normalized tissue titer by the average normalized titer in tumours infected with the corresponding virus.

### Statistical Analysis

Data were analysed by a two-tailed independent samples t-test between candidate VV and vvDD where applicable. Survival curves were evaluated with the log-rank test. Graphs were generated with the GraphPad™ Prism 5 Software (GraphPad Software Inc., La Jolla, Calif., USA). Data are presented as Mean±SEM.

### Example 2

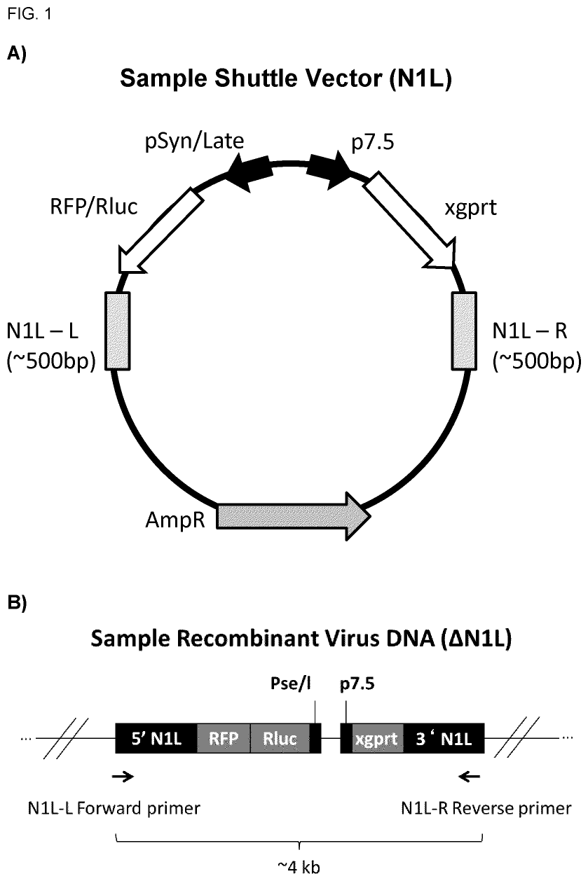

The methods described in Example 1 were used to generate Western Reserve (WR) vaccinia viruses (VVs) with a single gene deletion of one of candidate immunomodulatory VV genes (N1L, K1L, K3L, A46R, and A52R). The shuttle plasmids for poxvirus gene deletions were derived from a pre-existing plasmid called pVX-R2R-LUC. This vector was the same shuttle plasmid used to generate the vvDD-R2R-Luc as first described by Lun et al., herein incorporated by reference. Briefly, relevant features of the plasmid include 1) a VV synthetic early-late promoter upstream of a gene with RFP conjoined to Renilla luciferase via the foot-and-mouth disease virus 2A motif to facilitate bicistronic expression (i.e. referred to as R2R-Luc) and 2) a xanthine-guanine phosphoribosyltransferase (gpt) gene for viral selection under the VV p7.5 promoter, and 3) an ampicillin gene to enable bacterial selection (Lun et al., 2009). Sequences originally flanking the gene of interest (e.g. N1L) were cloned into the plasmid to flank the first 2 elements to create the shuttle plasmid (FIG. 1A) that was used to generate the desired VV deletion mutant with the DNA construct exemplified in FIGS. 1B and 18.

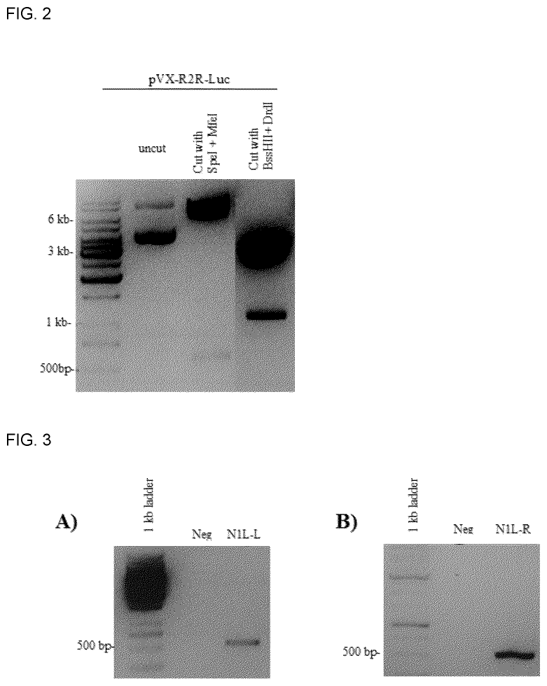

Before manipulating the plasmid, the plasmid preparation was confirmed to be pVX-R2R-Luc by restriction enzyme digestion with SpeI and MfeI, yielding the expected band sizes of 6 kb and 600 bp. A second double digestion with BssHII and DrdI yielded the expected sizes of two 2.8 kb and one 1 kb bands (FIG. 2).

The presently disclosed strategy was to delete poxvirus VV genes via homologous recombination by flanking the aforementioned marker genes in pVX-R2R-LUC with 300-500 bp sequences from either side of the candidate gene as determined by the Western Reserve (WR) VV genome sequence found in GenBank (Accession number: NC_006998.1, herein incorporated by reference). Shuttle plasmids were made to facilitate the deletion of the N1L, K1L, K3L, A52R and A46R VV gene. Using the ΔN1L VV shuttle plasmid, pVX-R2R-Luc, FIGS. 3-5 demonstrate the cloning process for generating the shuttle plasmid for the candidate VVs.

Primer designs to amplify DNA sequences on the left and right side of the candidate genes were derived from the VV genome sequence (Table 1) to facilitate PCR cloning. The PCR reaction was performed using the VJS6 (WR VV with a TK gene deletion) viral DNA as template. The sizes of the PCR products for the left and right flanking sequences were confirmed by agarose gel electrophoresis (FIG. 3). The DNA fragments were named based on the corresponding candidate gene followed by L or R depending on whether the sequence flanked the left or right side of the candidate gene, respectively. For example, the PCR product from amplifying the DNA sequence on the left side of the N1L gene was named “N1L-L”.

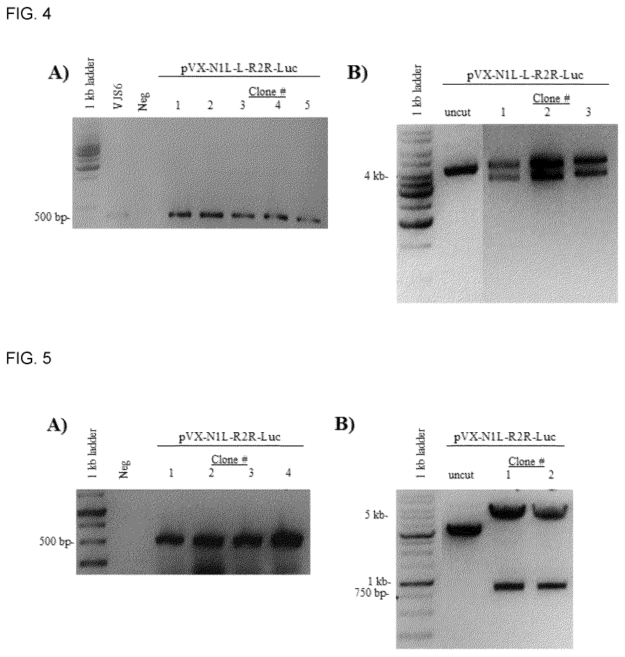

The corresponding left DNA fragments were inserted into pVX-R2R-Luc first. The plasmid was digested with MfeI and SpeI and the PCR fragments were digested with MfeI and NheI (which makes compatible ends to SpeI overhangs). After ligation, bacterial transformation and ampicillin selection, at least 3 colonies were tested for the presence of the correct insert via colony PCR (FIG. 4A) with corresponding primers to the PCR products made to insert into the vector. Positive clones were grown up for subsequent plasmid DNA isolation and further confirmed to contain the desired insert by restriction enzyme digestions (FIG. 4B). The resulting plasmids from bacterial transformation were named according to the format: “pVX-<gene name>-L-R2R-Luc”. For example, the pVX-R2R-LUC plasmid with an N1L-L insert was called “pVX-N1L-L-R2R-Luc”.

The plasmid preparation of one clone for each shuttle plasmid was used for subsequent cloning steps. The right side insert and the vectors with the corresponding left inserts were then double digested with PvuII and BssHII, ligated together, transformed into bacteria, and the final transformants were also confirmed by colony PCR first (FIG. 5A) and restriction enzyme digestion (FIG. 5B). All plasmid preparations were confirmed to be the desired clones by restriction enzyme digestion of unique or rare sites found in the insert and plasmid. The final shuttle plasmids to delete the N1L, K1L, K3L, A46R and A52R gene were named pVX-N1L-R2R-Luc, pVX-K1L-R2R-Luc, pVX-K3L-R2R-Luc, pVX-A46R-R2R-Luc, and pVX-A52R-R2R-Luc, respectively.

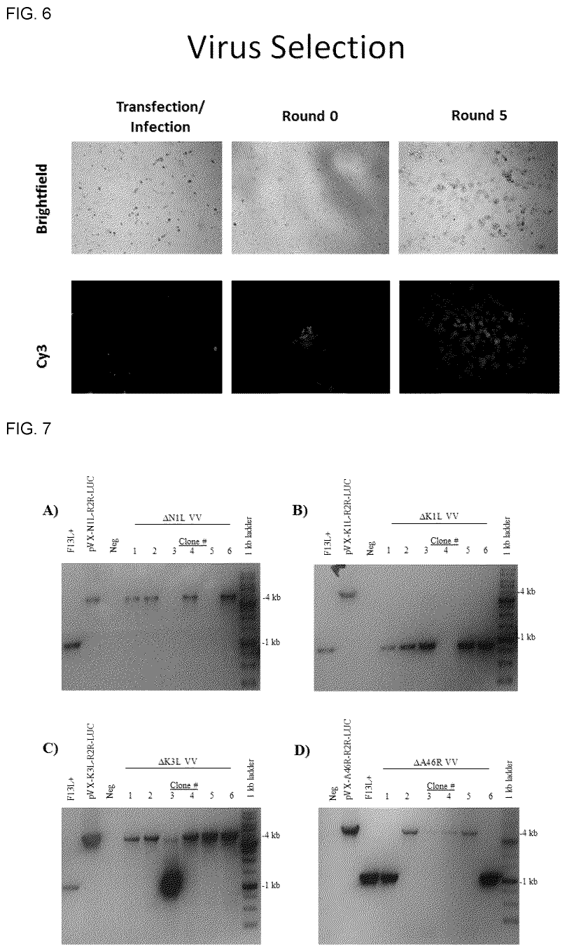

After confirmation by PCR and restriction enzyme digestion, the shuttle plasmids were amplified and transfected into F13L+(wildtype WR VV)-infected CV-1 cells. The cells were scraped up, serially diluted, and re-infected into a new monolayer of CV-1 cells growing in media supplemented with mycophenolic acid, hypoxanthine, and xanthine. Mycophenolic acid inhibits the salvage pathway for purine synthesis, specifically, the formation of guanine. If the F13L+ viruses undergo homologous recombination with the shuttle plasmid, in addition to the deletion of the candidate gene, the recombinant virus will express the RFP/Renilla luciferase marker gene and the Escherichia coli xgprt gene. The xgprt gene will allow the recombinant virus to circumvent the inhibition of mycophenolic acid and produce guanine to facilitate its replication while the wildtype virus will be able to infect cells, but not replicate (Mulligan & Berg, 1981). RFP expression was observed in single cells 72 h after the transfection/infection. After the initial re-infection of transfected cell suspension, RFP-expressing plaques were picked 72 hpi (Round 0) and re-infected into new monolayers of mycophenolic acid-treated CV-1 cells. Subsequent RFP-expressing plaques were picked for at least 5 rounds to dilute out the parental virus until the whole population was the desired recombinant virus as confirmed by PCR. Evidence of RFP expression after transfection and a sample RFP-expressing plaque at round 0 and 5 of selection are presented in FIG. 6.

The PCR confirmation of selected plaques as parental-free are were visualized via agarose gel electrophoresis and presented in FIG. 7. The primers used to amplify PCR products from the VJS6 DNA for insertion into pVX-R2R-Luc were used in these reactions. Specifically, the forward primer that amplified the left insert and the reverse primer of the right insert. For example, to confirm that the ΔN1L VV was parental free, the PCR reaction was run with the N1L-L forward and N1L-R reverse primers. In this way, if the virus is recombinant with the desired interruption of the open reading frame (ORF) of the candidate VV gene, the expected band as a result of the inserted marker genes was approximately 4 kb. If parental virus F13L+ was still present in the recombinant virus sample, the PCR product will be the combined length of both PCR inserts used for cloning and the wildtype candidate gene sequence left in between (approximately 1 kb). Although it is possible that the PCR reaction can yield the recombinant DNA product, a length of 4 kb is very difficult to achieve in a PCR reaction with standard Taq polymerase, especially with a crude preparation of viral DNA template. Furthermore, the goal of the PCR is to confirm the absence of parental virus. Hence, clones were considered fit for further use based primarily on the absence of the ˜1 kb PCR product from the candidate gene in a wildtype WR VV. As expected, some PCR reaction neither yielded the 4 kb nor the 1 kb band such as the ΔK1L VV clone #4 (FIG. 7B). The plaque from this clone was still amplified into viral stocks because the viral RFP expression and growth in the presence of mycophenolic acid indicates the presence of the desired virus. For the other viruses, the following clones were used to amplify the desired recombinant viruses: clone #4 of ΔN1L VV (FIG. 7A), clone #6 of ΔK3L VV (FIG. 7C), and clone #2 of ΔA46R VV (FIG. 7D).

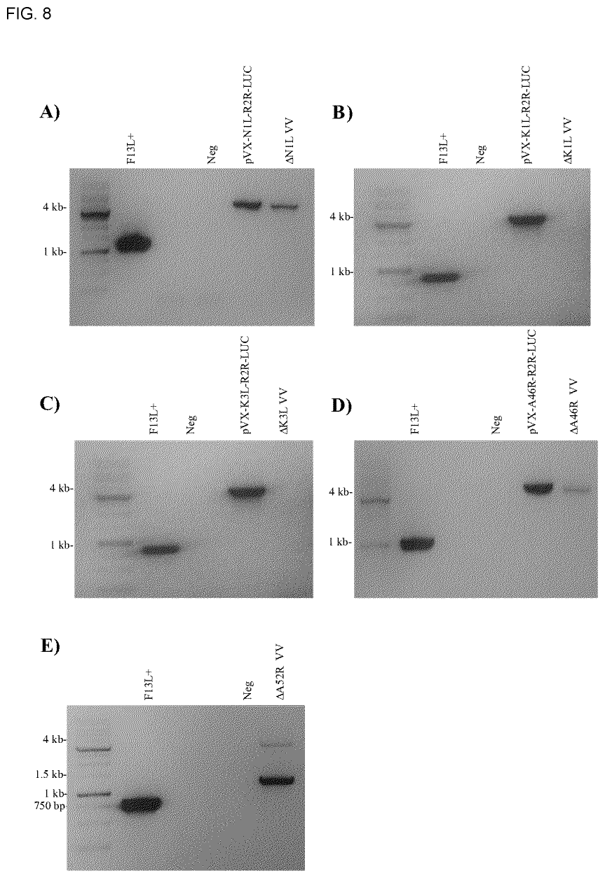

During and after the process of amplifying the candidate VVs into viral stocks, the same PCR reaction described in FIG. 8 was conducted to further confirm that absence of parental WR VV virus before further use for experimental assays. If there were any parental virus left in the selected plaques tested above that did not produce a 1 kb band due to, for example, too low amounts of parental virus DNA template compounded by inhibitory factors within the crude DNA preparation, the amplification process will have also amplified the parental virus and produced a 1 kb band in the PCR reaction by the end of the viral stock production. The stock of ΔA52R VV was also tested here. It was confirmed that all stocks of candidate VVs were free from parental WR VV (FIG. 8).

After confirmation that the virus stocks were free from parental virus, the candidate VVs were tested in vitro as described below.

### Example 3

Compare Candidate VV Deletion Mutants to vvDD in Terms of Viral Replication, Cytotoxicity, and Spread In Vitro in Colon and Ovarian Cancer Cell Lines.

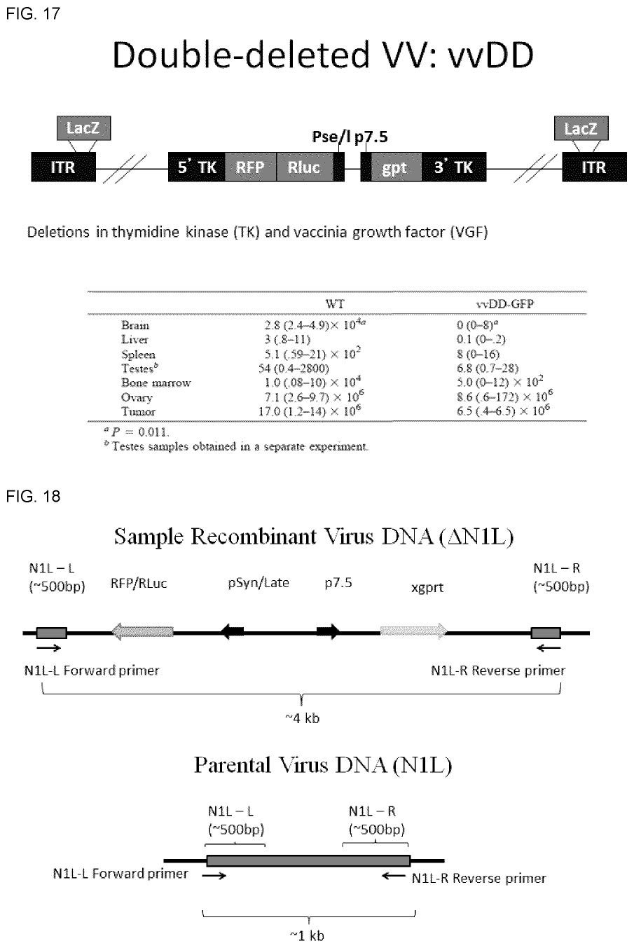

The potential of candidate deletion mutants described in Example 2 as an oncolytic agent was evaluated by testing the capability of the constructs for viral replication, tumour-killing, and viral spread compared to a clinical oncolytic virus such as vvDD (see FIG. 17 for a schematic of vvDD). Three cell lines were used: MC38 murine colon carcinoma, DLD-1 human colon carcinoma, and A2780 human ovarian colon carcinoma. These cell lines were chosen to correspond with the cell lines used in the mouse models of peritoneal carcinomatosis (PC) described subsequently for the in vivo evaluation of VV deletion mutants.

Candidate VV Replication is Similar or More Efficient than vvDD in Monolayers of Colon and Ovarian Cells.

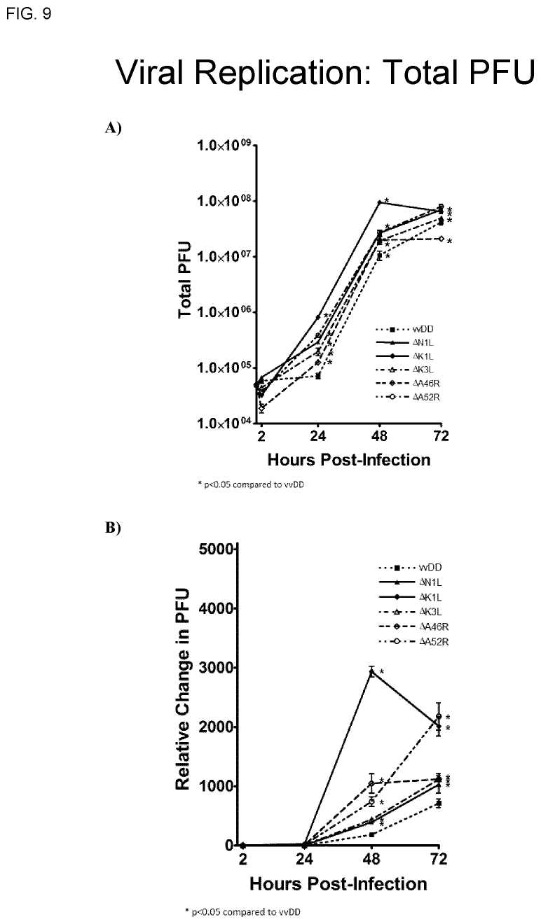

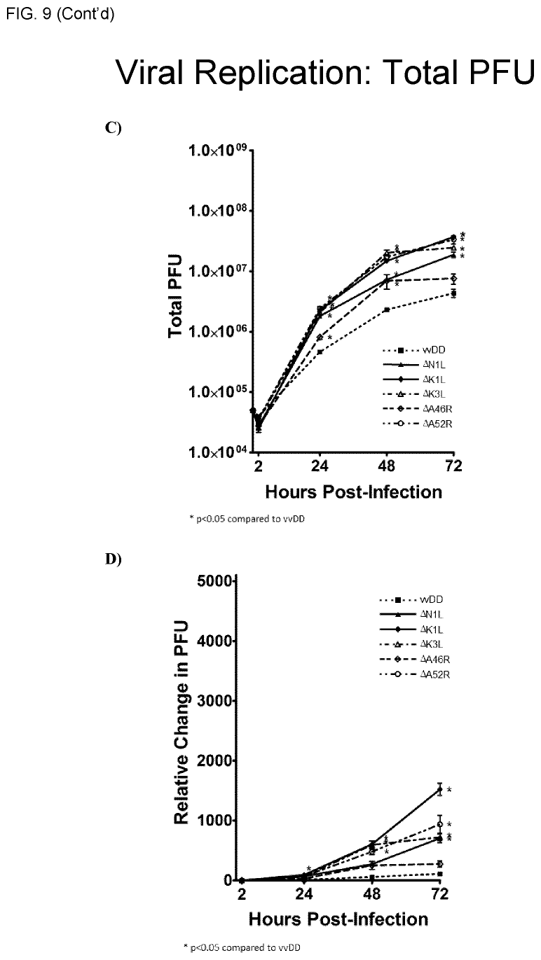

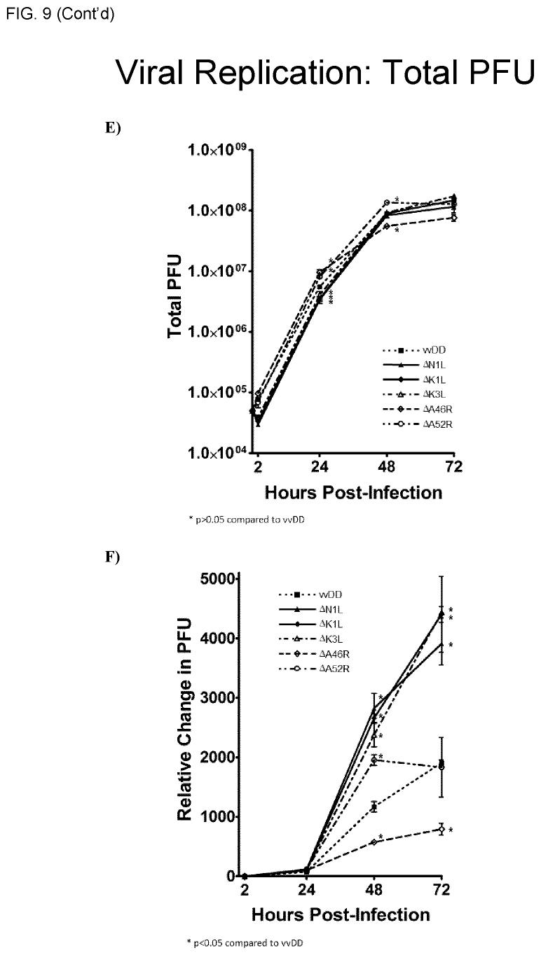

The viral replication of candidate VV deletion mutants and vvDD in cancer cells was assessed by infecting monolayers of MC38 (FIGS. 9A and B), DLD-1 (FIGS. 9C and D) or A2780 (FIGS. 9E and F) at a low MOI of 0.1. Infected monolayers were harvested and stored for subsequent virus quantification by plaque assay at baseline (or 2 hours after the pre-infection process), 24, 48, and 72 hpi (FIG. 9).

VV replication for vvDD and VV deletion mutants in all cell lines resulted in a 2-3 log-fold increase in total viral particles by 72 hpi. In general, all candidate VVs exhibited similar or better viral replication compared to vvDD in both colon cancer cell lines at peak viral titers. The same can be concluded for most candidate VVs within A2780 cells except the ΔA46R VV. However, the replication efficiency of all tested VVs in A2780 cells were generally higher compared to the other two colon carcinoma cells [range of mean viral fold change compared to baseline at 72 hpi: 712.15-2177.47 (MC38), 109.43-1520.41([DLD-1), and 791.99-4405.20 (A2780)]. The ΔK1L VV shows the most efficient replication ability across all cell lines, with the average fold change in replication being 2.8, 13.9, and 2.3 times higher than the average fold change of vvDD.

Candidate VV Spread is Similar or Better than vvDD in Monolayers of Colon and Ovarian Carcinoma Cells.

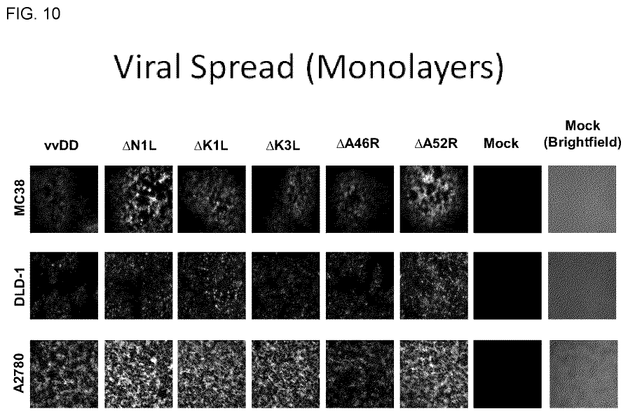

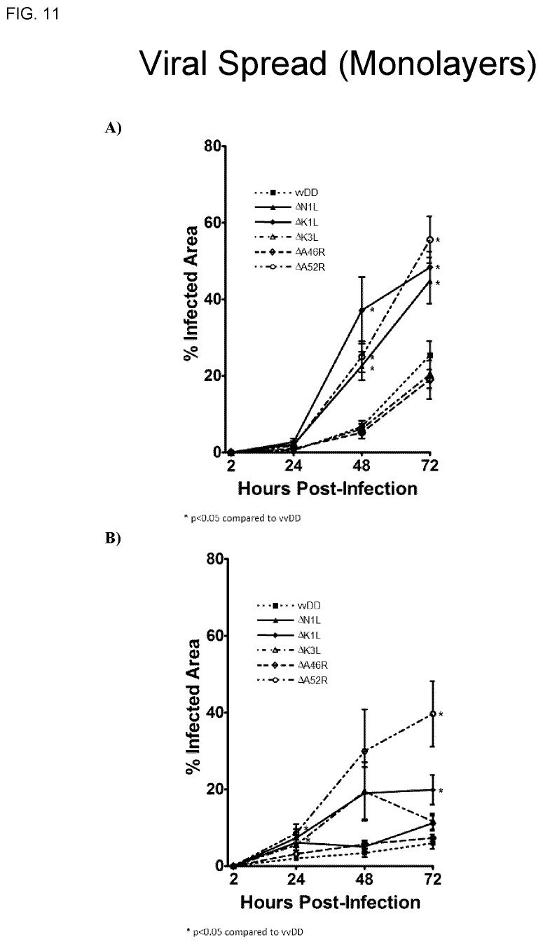

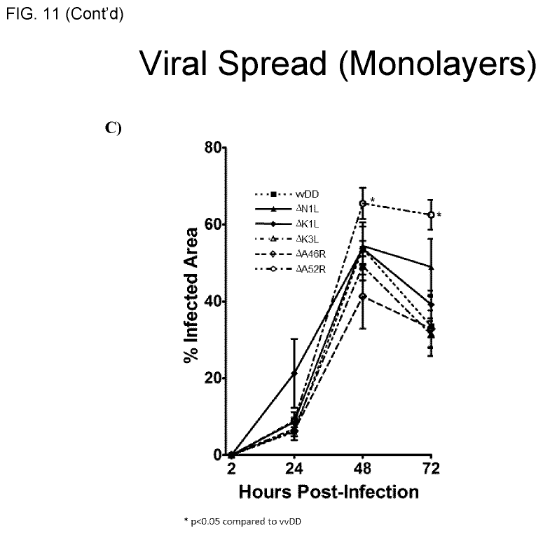

The viral spread of the candidate VV deletion mutants were compared to vvDD in monolayers of colon and ovarian cell lines infected at an MOI of 0.1 by fluorescence microscopy. Differential interference contrast (DIC) images and fluorescent images with the Cy3 lens were taken at 2 (baseline), 24, 48, and 72 hpi. Images at peak viral spread based on RFP expression are presented: 72 hpi of MC38 and DLD-1 cells and 48 hpi of A2780 cells (FIG. 10). Viral spread quantification was based on the area of RFP expression relative to the total area of the image. The values presented represent a combination of two independent experiments (FIG. 11).

The viral spread of all candidate VVs was similar or better than vvDD in all cell lines tested, as confirmed by both visual assessment and quantitative analysis. Statistical significance was achieved in MC38 cells for these viruses, and visual assessment of viral spread confirms the quantified trend of superior spread in ΔK1L VV, ΔN1L VV, and ΔA52R VV in all cell lines. The best performer, ΔA52R VV, demonstrated consistently significant advantage in viral spread in all cell lines, infecting approximately half of all cell lines at its peak [% infected area: 55.57%±6.076% (MC38), 39.68%±8.51% (DLD-1), 65.48%±4.06% (A2780). As such, mean ΔA52R VV spread was 11.75% to 33.72% better relative to vvDD at peak time points in all tested cell lines.

Candidate VVs are Similarly or More Cytotoxic to Monolayers of Colon and Ovarian Carcinoma Cells Compared to vvDD.

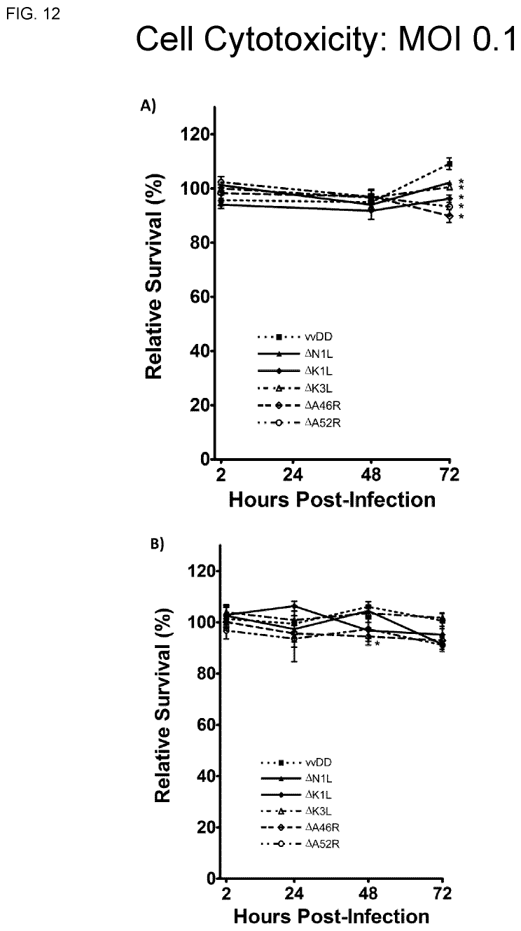

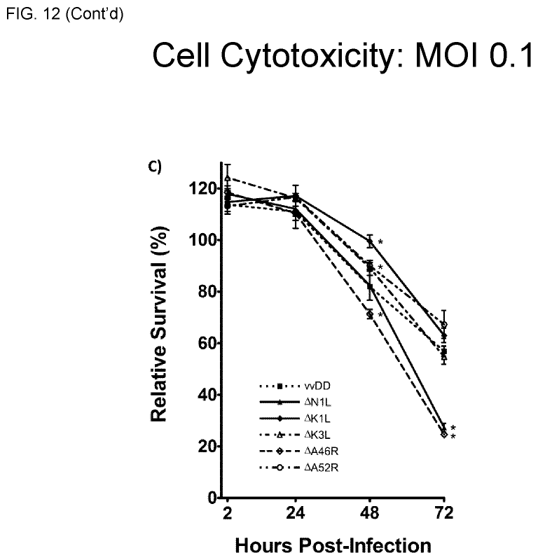

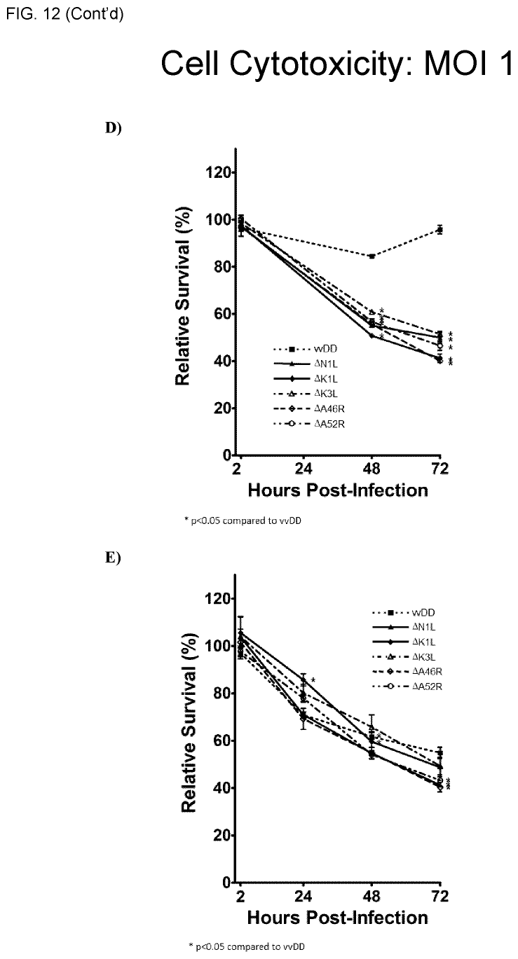

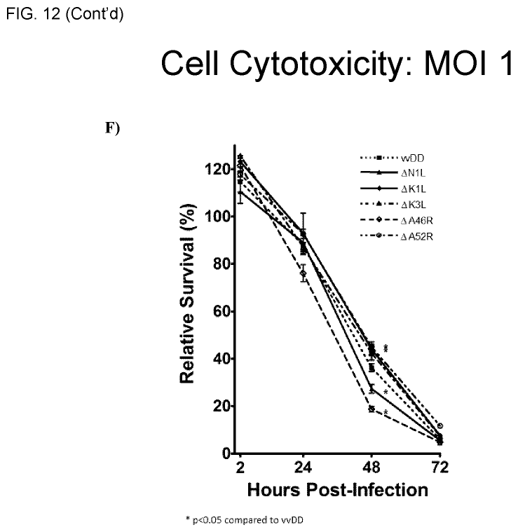

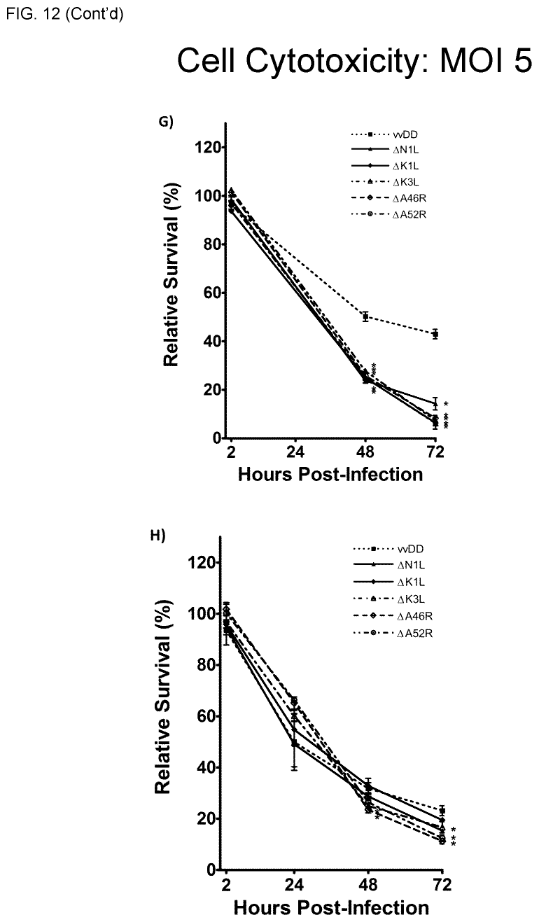

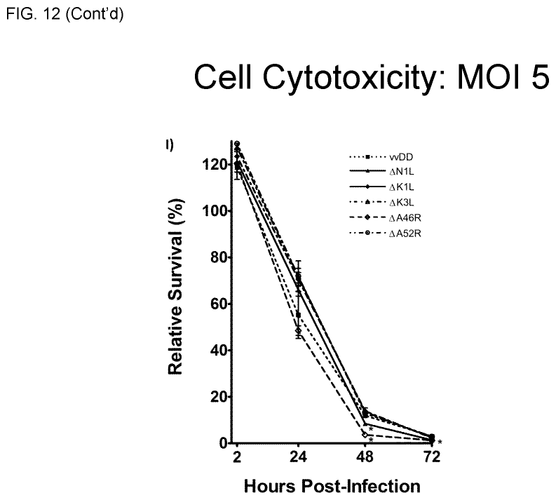

The cytotoxicity of candidate VVs were compared to vvDD at multiple doses (MOI 0.1, 1, and 5) and multiple time points (2, 24, 48, and 72 hpi) in monolayers of colon and ovarian cell lines. Cell viability was measured via the colorimetric MTS ([3-(4,5-dimethylthiazol-2-yl)-5-(3-carboxymethoxyphenyl)-2-(4-sulfophenyl)-2H-tetrazolium inner salt) assay. Percent viability is presented as absorbance values relative to mock-treated cells (FIG. 12).

Similar to the results of the viral replication experiments, the cytotoxicity of the candidate VVs is either equal or higher in the colon cancer cell lines, MC38 and DLD-1, compared to vvDD. The difference in cytotoxicity of all candidate VVs compared to vvDD was the most dramatic in MC38 cells, where candidate VV treatment at 72 hpi at MOI of 1 reduced the mean cell viability relative to mock controls by at least 1.92 times compared to vvDD (range: 1.92 to 2.39 times, p<0.05). Some of the constructed VVs were statistically less toxic to A2780 cells than vvDD, however, the VVs were still very effective at killing A2780 cells. Dramatically, cytotoxicity of all candidate VVs and vvDD towards A2780 cells demonstrated significant cytopathic effect at a low MOI of 0.1 by 72 hpi (mean % viability: 24.61% to 63.00%). The cytotoxicity against all tumour cell lines of all candidate VVs was similar by 72 hpi. Among them, the ΔA46R VV was the most cytotoxic [mean % viability at MOI 1: 40.07%0.29% (MC38), 40.32%±2.00% (DLD-1), and 4.75%+1.08% (A2780)].

Candidate VVs Exhibit Equal or Better Viral Spread in Tumour Spheroids Compared to vvDD.

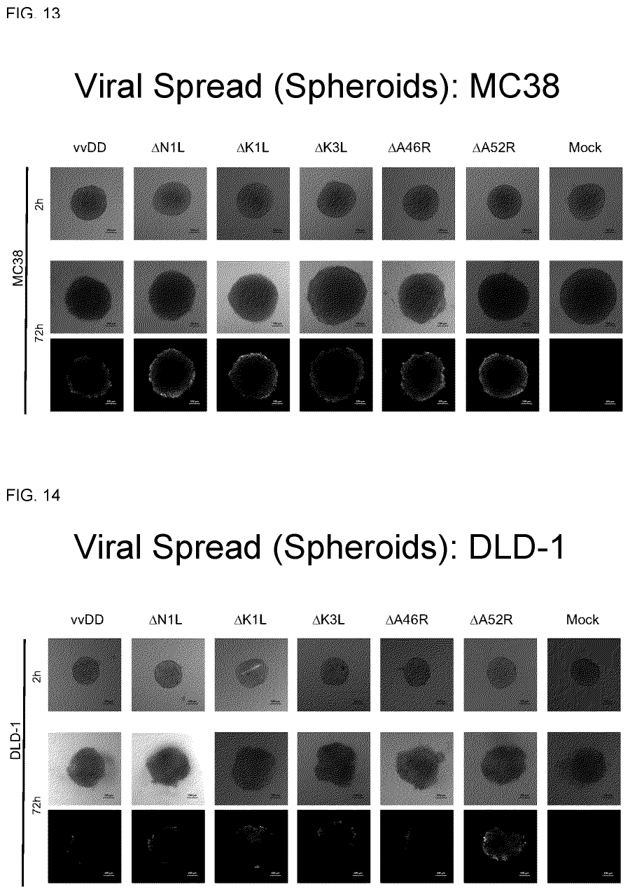

Tumour spheroids mimic the 3D structure and microenvironment of a tumour and allow for a more relevant and realistic in vitro investigation (Casagrande et al., 2014) of the candidate VVs. In this study, the MC38 and DLD-1 spheroids were formed, infected at an MOI of 2, and viral spread was observed through the Cy3 lens of a confocal microscope to track virally-induced RFP expression throughout the spheroid over time. The spheroid diameter was approximately 250-300 μm at the time of infection. The images presented are representative fluorescent images approximately halfway into the spheroid (˜130 μm) (FIGS. 13 and 14). A snapshot of the spheroid with the brightfield lens was also taken to calculate spheroid volume using an equation that assumed a spherical/elliptical shape. Tumour volume was only calculated for MC38 spheroids because DLD-1 spheroids tend to become very irregular as a result of infection. A2780 cells were not used to make spheroids in this study because tumour spheroid generation with PolyHEMA plates did not yield the desired uniform, compact cell aggregates which could survive the aspiration step during infection. Instead, the A2780 cells formed loose cell aggregates, which is consistent with literature (Casagrande et al., 2014).

Viral spread into tumour spheroids were qualitatively evaluated via viral RFP marker expression. The viruses varied in their ability to penetrate into the centre of the 3D structures. Small amounts of RFP expression associated with vvDD infection could be seen at 130 μm deep into MC38 spheroids at 72 hpi. In contrast, the ΔN1L VV, ΔK1L VV, ΔA46R VV, and ΔA52R VV infected at least most of the rim and demonstrated some penetration into the MC38 spheroids. The DLD-1 spheroids were more resistant to infection as vvDD and most candidate VVs resulted in small dispersed foci of RFP by 72 hpi. However, the ΔA52R VV was able to infect a larger portion of the DLD-1 spheroids. Out of all the tested viruses, ΔA52R VV could spread most efficiently in both MC38 and DLD-1 spheroids.

Candidate VVs and vvDD are Cytotoxic to Tumour Spheroids.

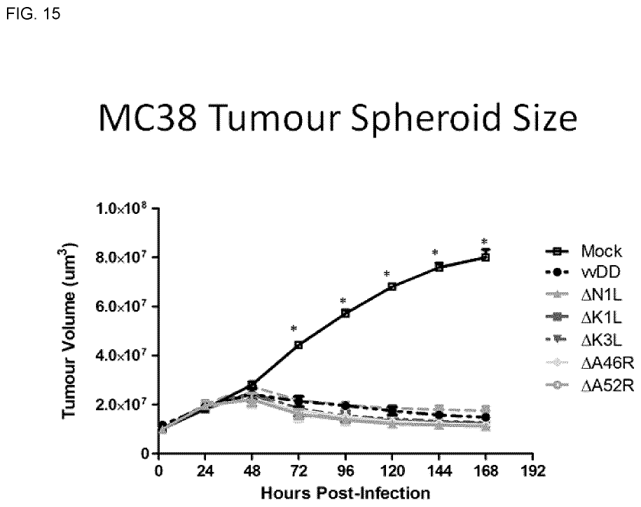

The cytotoxicity of vvDD and candidate VVs towards MC38 and DLD-1 spheroids were evaluated by a clonogenic assay at 96 hpi of spheroids that were infected at an MOI of 2. This time point was chosen based on the MC38 tumour volume calculated from the brightfield images taken from confocal microscopy. Specifically, at 96 hpi for MC38 spheroids, the decrease in tumour spheroid volume started to plateau (FIG. 15). Hence, both MC38 and DLD-1 spheroids were disassociated at 96 hpi for the clonogenic assay.

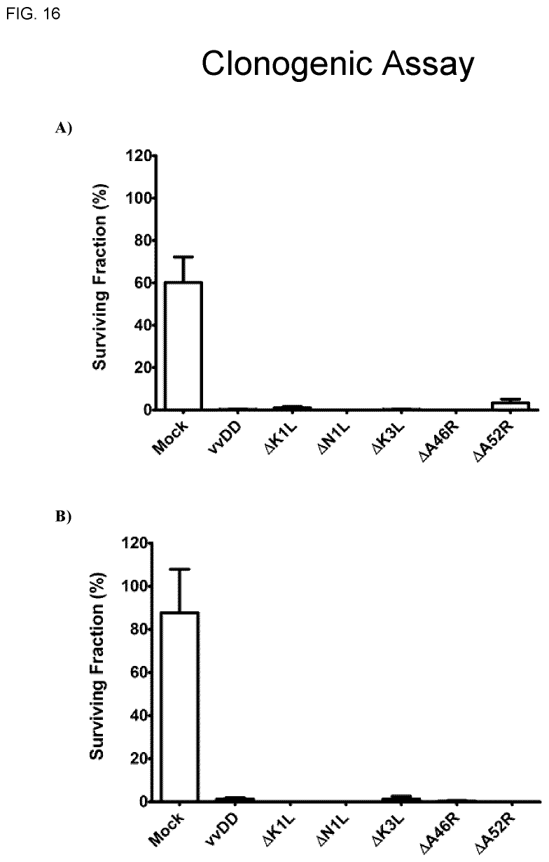

The percent surviving fraction of virus-treated MC38 spheroids was low or undetectable (range of mean surviving fraction 0-3.3%). MC38 spheroids treated with ΔA46R or ΔN1L VV did not yield a detectable surviving fraction (FIGS. 16A and B). Similarly, surviving fraction of DLD-1 spheroids treated with all viruses were very low to undetectable (range of mean surviving fraction: 0-1.3%). DLD-1 spheroids treated with ΔK1L VV, ΔN1L VV, and ΔA52R VV did not result in a detectable surviving fraction (FIGS. 16C and D). In contrast, many cells survived in mock-treated spheroids (mean surviving fraction MC38: 60.2%±12.0%, DLD-1: 87.7%±20.2%).

### In Vivo Studies

Three candidate VVs were chosen for initial in vivo investigation. Candidate VVs were assessed against vvDD for viral replication, spread, and tumour cytotoxicity in MC38, DLD-1, and A2780 cells and results are summarized in Table 2, ranking the performance of each virus starting with the best performer on top. ΔK1L VV demonstrated at the greatest viral replication, ΔA46R VV was the most cytotoxic, and ΔA52R demonstrated the most viral spread in tumours. These three recombinant VVs were tested in in vivo models of cancer, in particular, established models of peritoneal carcinomatosis (PC).

Assess the Tumour-Selectivity and Anti-Tumour Efficacy of Candidate VV Treatment Compared to vvDD at Maximum Tolerable Doses (MTD) in Nude and Immunocompetent Mice.

To further investigate the potential of the presently disclosed candidate VVs as an oncolytic virus, the best performers from in vitro assays were tested in nude (NU/NU) and immunocompetent mice (C57BL/6). A maximum tolerable dose (MTD) was determined for each candidate VV and used for subsequent investigation. For both the biodistribution and tumour survival studies, tumour generation was performed via IP injection of either DLD-1 human colon carcinoma cells or A2780 human ovarian carcinoma cells into NU/NU mice or MC38 murine colon carcinoma cells into immunocompetent C57BL/6 mice. These mouse models are established models of peritoneal carcinomatosis (PC).

Before assessing the anti-tumour efficacy of each candidate VV treatment, a treatment dose must be determined for the IP treatment. Non-tumour-bearing mice were injected IP at different doses and followed for toxicity. Common adverse events of virus treatment in nude mice included pox formation, usually on its paws or tail, and weight loss. However, in C57BL/6 mice, only weight loss, if at all, was observed after virus treatment.

When C57BL/6 mice were injected at the same dose as the established vvDD IP dose of 10⁹ pfu, the mice treated with candidate VVs reached endpoint within a week. Subsequent studies tested doses one at a time and observed for at least 30 days or until the first death before the initiation of another study with an adjusted dose. Doses were increased or decreased by 0.5 log-fold in the next study accordingly. For most of the dose titration experiments, no mock group was used. Instead, vvDD was injected at the same dose as the candidate VVs for each study. According to literature, vvDD was shown to be non-toxic to mice even at an IP dose of 109, though transient weight loss after injection was observed (McCart et al., 2001; Ottolino-Perry et al., 2014). Thus, no deaths were expected from the vvDD-treated group, especially at lower doses. Hence, the vvDD group served as 1) a negative control similar to mock, and 2) the treatment group to compare to other virus treatment groups for in vivo toxicity since the vvDD treatment could still cause weight loss.

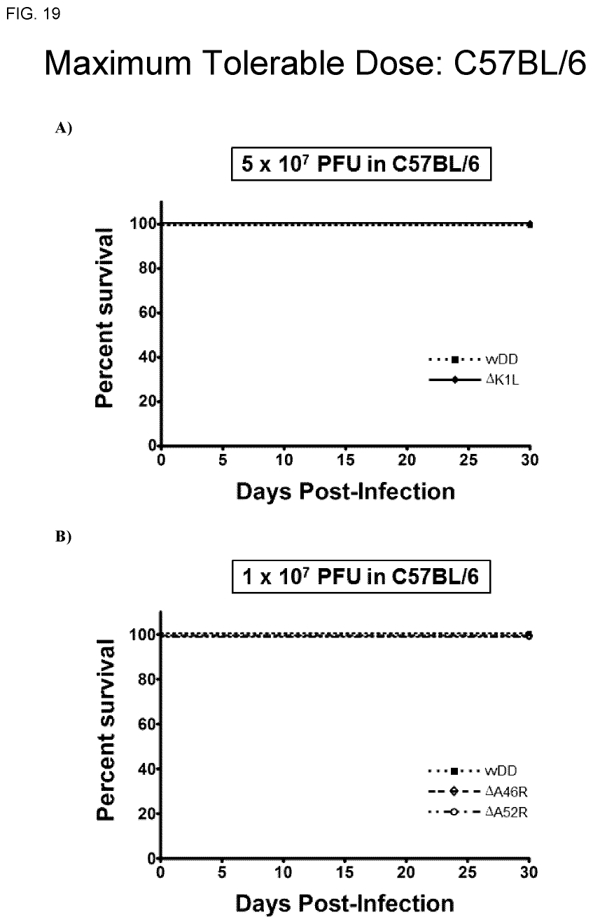

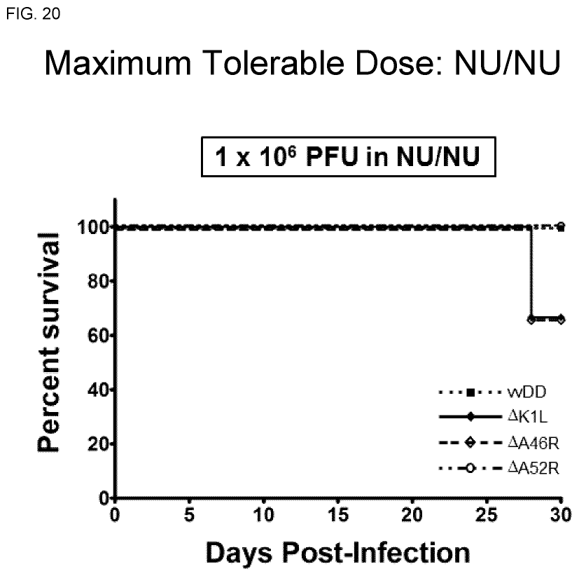

MTDs were determined by injecting varying doses of VV IP into non-tumour-bearing mice. Doses at which mice survived for 4 weeks or more were deemed the MTD and used for subsequent tumour survival studies. For immunocompetent C57BL/6 mice, the IP MTD for the ΔK1L VV was 5×10⁷ pfu, while the MTD of ΔA46R VV and ΔA52R VV was 1×10⁷ pfu (FIGS. 19 and 20). The IP treatment dose for the tumour survival studies of all candidate VVs in nude mice was 10⁶ pfu. In contrast, the established IP dose of vvDD is 10⁹ pfu, where only transient weight loss after injection is observed (McCart et al., 2001; Ottolino-Perry et al., 2004).

Candidate VVs have Improved or Similar In Vivo Tumour-Selectivity and Anti-Tumour Effect Compared to vvDD

The tumour efficacy and tumour-selectivity of IP-delivered candidate VVs, vvDD, and mock treatment were compared in tumour-bearing models of PC. Specifically, a syngeneic model of C57BL/6 mice bearing IP MC38 tumours and xenograft models of nude (NU/NU) mice bearing IP A2780 or DLD-1 tumours were used. Mice were injected IP with tumour cells and treated with VVs 12 days later at indicated doses.

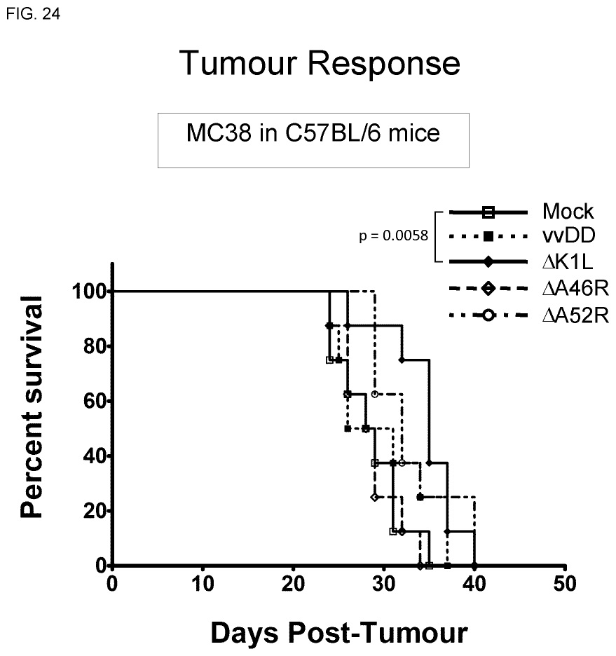

Despite significantly lower treatment doses, candidate VV deletion mutants improved survival similar to or better than vvDD with respect to mock-treated controls. In the MC38 tumour-bearing C57BL/6 syngeneic model (FIG. 24), both vvDD- and ΔA46R VV-treated mice had a similar survival time compared to mock-treated mice. A significant improvement in median survival time compared to mock-treated mice was observed in ΔK1L-treated mice (35 days vs 28.5 days, p=0.0058), but ΔA52R VV-associated median survival was also longer than vvDD or mock-treatment (32.5 days).

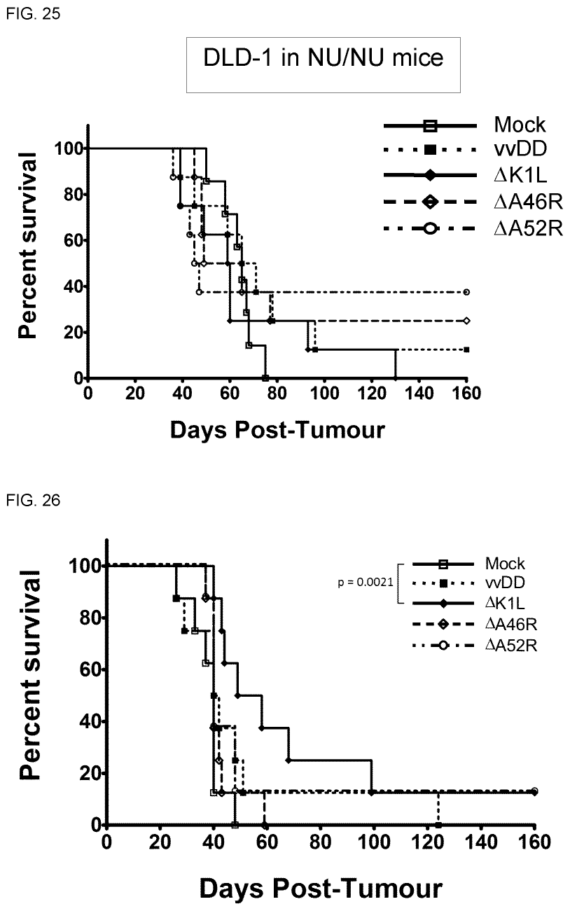

The efficacy of the candidate VVs was also similar or better than vvDD in xenograft models of PC despite a 1000-fold lower treatment dose. In DLD-1 tumour-bearing NU/NU mice (FIG. 25), long-term survival until the end of the experiment at 160 days post infection (dpi) was observed in 12.5%, 25%, and 37.5% of vvDD, ΔA46R VV and ΔA52R VV treated mice, respectively. In A2780 tumour-bearing mice (FIG. 26), ΔK1L VV treatment significantly improved median survival time compared to mock-treated mice (53.5 days vs 40 days, p=0.0021). A 12.5% long-term survival rate was also associated with vvDD, ΔK1L VV, and ΔA52R VV treatment.

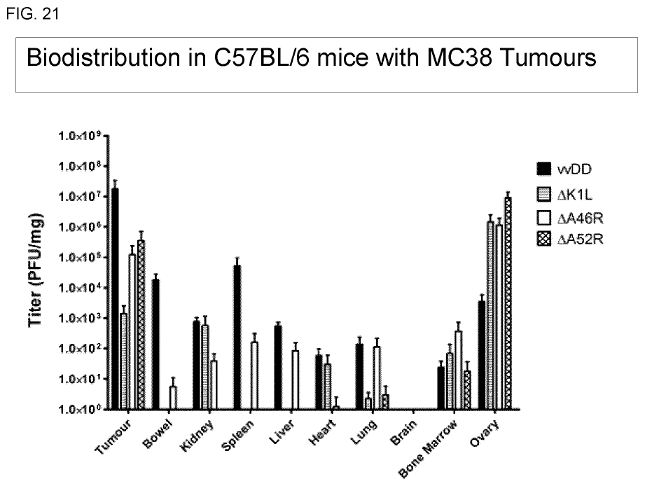

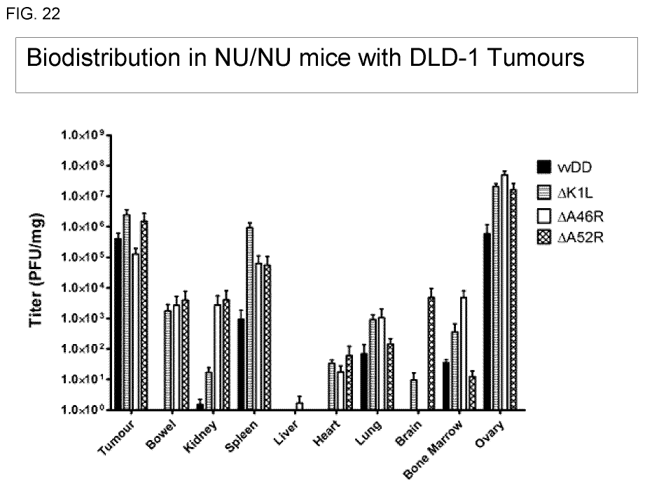

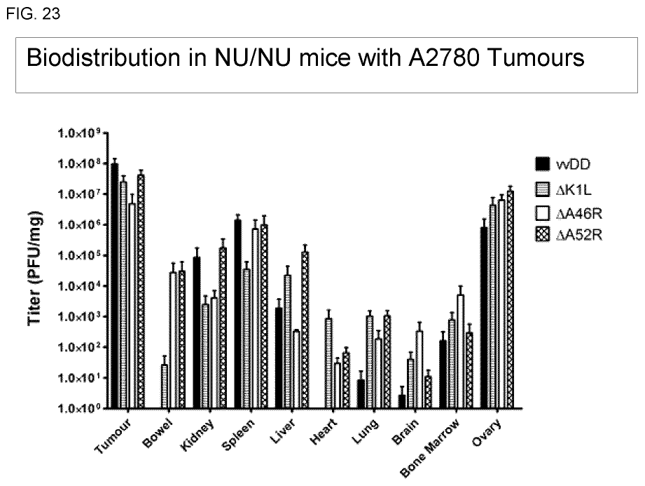

VV replication preferentially targeted the tumour and the ovaries in all cancer mouse models tested (FIGS. 21-24). In C57BL/6 mice bearing MC38 tumours (FIG. 21), 2-4 log-fold less candidate VV deletion mutant was detected in the tumour compared to vvDD, possibly partially due to the different doses. With the exception of the ovaries, all viruses had little to no infectious particles in other non-tumour tissues compared to tumour. Impressively, ΔA52R VV was undetectable in the bowel, spleen, liver, heart, and brain. In DLD-1 tumour-bearing NU/NU mice (FIG. 22), the viral load of candidate VVs and vvDD found in tumours was more similar (range: 1.3×10⁵ pfu/mg-1.5×10⁶ pfu/mg compared to 4.1×10⁵ PFU/mg). In general, the concentration of infectious particles of all candidate VV deletion mutants and vvDD was lower in non-tumour tissues, except the ovary, compared to tumour. In A2780 tumour-bearing NU/NU mice (FIG. 23), up to 10 times more than the amount of virus injected into each mouse was found per milligram of tumour for all viruses. In addition, the viral load in all non-tumour tissues was at least 10 times lower than the viral load found in corresponding tumours.

Candidate VVs and vvDD Treatment are Associated with Increased Immune Cells Infiltration into Tumours

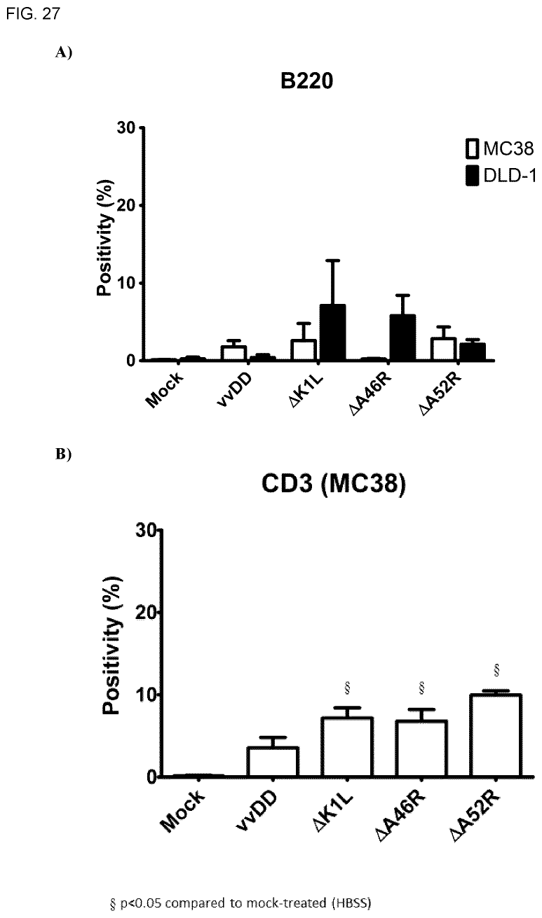

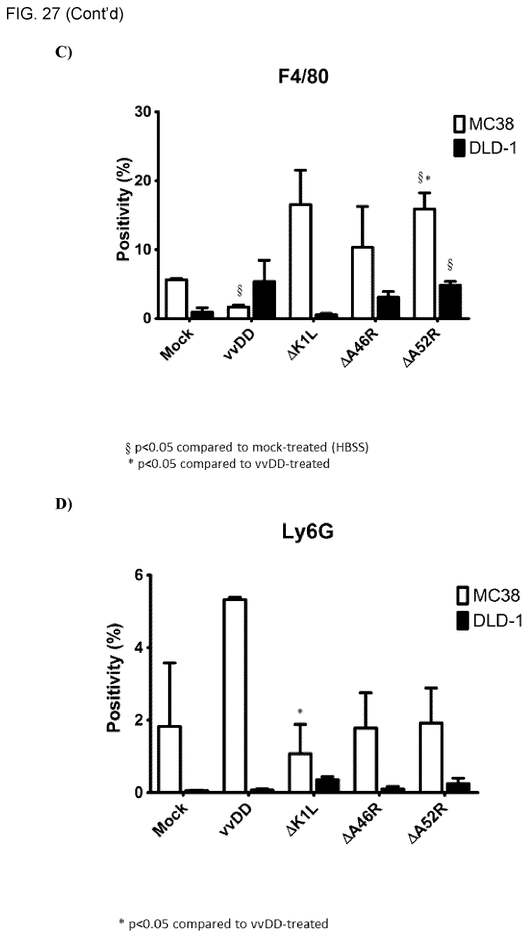

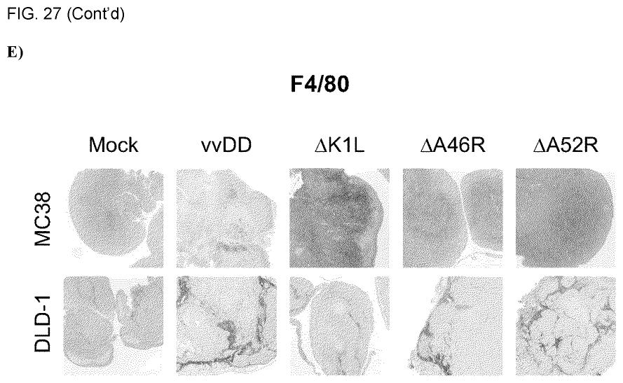

A portion of the tumours from the biodistribution studies in the MC38 and DLD-1 tumour mouse models were used for immunohistochemistry and stained for B220 (B-cells), CD3 (T-cells), F4/80 (macrophages), and Ly6G (neutrophils). The percent positively-stained pixels in the tumour sections were quantified using the Imagescope Positive Pixel Algorithm (FIG. 27). There were no significant differences in B220 staining between all treatment groups for both tumour models (FIG. 27A) whereas all candidate VVs were associated with increased T-cell infiltration into MC38 tumours (ΔK1L VV: 7.18%±0.78%; ΔA46R VV: 6.79° %0.81%; ΔA52R VV: 9.96%±0.30%) compared to mock-treatment (0.15%±0.06%), but not vvDD (3.56%±0.89%; FIG. 27B). Moreover, the percent F4/80 staining in MC38 tumours treated with ΔA52R VV was at least 3× times higher than both mock- and vvDD-treated tumours. Similarly, ΔA52R VV-treatment in DLD-1 tumours was associated with increased mean percent F4/80 staining than mock-treatment (FIGS. 27C and E). In contrast, MC38 tumours treated with ΔK1L VV had decreased mean percent Ly6G staining of 1.070%0.47% compared to 3.57%±1.01% associated with vvDD treatment (FIG. 27D).

TABLE 3
VV Immunomodulatory Genes

| Gene | Role |
|---|---|
| A52R | Inhibits TLR signalling through TRAF6 and |
| | IRAK2, induce IL-10 expression |
| A46R | Inhibits TLR signalling through MyD88 and TRIF |
| N1L | Inhibits NF-κB through IKKβ and inhibits |
| | apoptosis |
| K1L | Host range protein that also inhibits NF-κB by |
| | preventing IκB phosphorylation |
| K3L | Prevents phosphorylation of e1F2α, inhibits PKR |

All publications, patents and patent applications mentioned in the specification are indicative of the level of those skilled in the art to which this disclosure pertains. All publications, patents and patent applications are herein incorporated by reference to the same extent as if each individual publication, patent, or patent application was specifically and individually indicated to be incorporated by reference. The mere mentioning of the publications, patents and patent applications does not necessarily constitute an admission that they are prior art to the instant disclosure.

Although the foregoing disclosure has been described in some detail by way of illustration and example for purposes of clarity of understanding, it is to be understood that the disclosure is not limited to the disclosed example. To the contrary, the disclosure is intended to cover various modifications and equivalent arrangements included within the spirit and scope of the appended claims.

### CITATIONS FOR REFERENCES REFERRED TO IN THE SPECIFICATION

Altschul, S. F., Gish, W., Miller, W., Myers, E. W. and Lipman, D. J. (1990). Basic local alignment search tool. Journal of molecular biology 215, 403-410.
Altschul, S. F., Madden, T. L., Schaffer, A. A., Zhang, J., Zhang, Z., Miller, W. and Lipman, D. J. (1997). Gapped BLAST and PSI-BLAST: a new generation of protein database search programs. Nucleic acids research 25, 3389-3402.
Antonio Chiocca, E. (2002). Oncolytic Viruses. Nature Reviews Cancer 2 (12): 938-50. doi: 10.1038/nrc948.
Casagrande, N., Celegato, M., Borghese, C., Mongiat, M., Colombatti, A. and Aldinucci, D. (2014). Preclinical activity of the liposomal cisplatin lipoplatin in ovarian cancer. Clinical Cancer Research, 20(21), 5496-5506. http://doi.org/10.1158/1078-0432. CCR-14-0713
Heo, Jeong, Reid, T., Ruo, L., J Breitbach, C. J., Rose, S., Bloomston, M., Cho, M., Lim, H. Y., Chung, H. C., Kim, C. W., Burke, J., Lencioni, R., Hickman, T., Moon, A., Lee, Y. S., Kim, M. K., Daneshmand, M., Dubois, K., Longpre, L., Ngo, M., Rooney, C., Bell, J. C., Rhee, B. G., Patt, R., Hwang, T. H. and Kim, D H. (2013). Randomized Dose-Finding Clinical Trial of Oncolytic Immunotherapeutic Vaccinia JX-594 in Liver Cancer. Nature Medicine, 19(3), 329-36. http://doi: 10.1038/nm.3089.
Karlin, S. and Altschul, S. F. (1990). Methods for assessing the statistical significance of molecular sequence features by using general scoring schemes. Proceedings of the National Academy of Sciences 87, 2264-2268.
Karlin, S. and Altschul, S. F. (1993). Applications and statistics for multiple high-scoring segments in molecular sequences. Proceedings of the National Academy of Sciences 90, 5873-5877.
Lun, X. Q., Jang, J. H., Tang, N., Deng, H., Head, R., Bell, J. C., Stojdl, D. F., Nutt, C. L., Senger, D. L., Forsyth, P. A. and McCart, J. A. (2009). Efficacy of systemically administered oncolytic vaccinia virotherapy for malignant gliomas is enhanced by combination therapy with rapamycin or cyclophosphamide. Clinical Cancer Research, 15(8), 2777-2788. http://doi.org/10.1158/1078-0432. CCR-08-2342
McCart, J. A., Ward, J. M., Lee, J., Hu, Y., Alexander, H. R., Libutti, S. K., Moss, B., and Bartlett, D. L. (2001). Systemic Cancer Therapy with a Tumor-Selective Vaccinia Virus Mutant Lacking Thymidine Kinase and Vaccinia Growth Factor Genes. Cancer Research 61: 8751-8757.
Mulligan, R. C. and Berg, P. (1981). Selection for animal cells that express the Escherichia coli gene coding for xanthine-guanine phosphoribosyltransferase. Proceedings of the National Academy of Sciences of the United States of America, 78(4), 2072-2076.
Myers, E. W. and Miller, W. (1988). Optimal alignments in linear space. Computer applications in the biosciences: CABIOS 4, 11-17.
Ottolino-Perry, K., Tang, N., Head, R., Ng, C., Arulanandam, R., Angarita, F. A., Acuna, S. A., Chen, Y., Bell, J., DaCosta, R. S. and McCart, J. A. (2014). Tumor vascularization is critical for oncolytic vaccinia virus treatment of peritoneal carcinomatosis. International Journal of Cancer. Journal International Du Cancer, 134(3), 717-730. http://doi.org/10.1002/ijc.28395
Roper, R. L. and Moss, B. (1999). Envelope formation is blocked by mutation of a sequence related to the HKD phospholipid metabolism motif in the vaccinia virus F13L protein. J. Virol. 73: 1108-1117
Zeh, H. J., Downs-Canner, S., McCart, J. A., Guo, Z. S., Rao, U. N. M., Ramalingam, L., Thorne, S. H., Jones, H. L., Kalinski, P., Wieckowski, E., O'Malley, M. E., Daneshmand, M., Hu, K., Bell, J. C., Hwang, T. H., Moon, A., Breitbach, C. J., Kirn, D. H. and Bartlett, D. L. (2015). First-in-Man Study of Western Reserve Strain Oncolytic Vaccinia Virus: Safety, Systemic Spread, and Antitumor Activity. Molecular Therapy, 23(1), 202-14. http://doi:10.1038/mt.2014.194.
U.S. Pat. No. 5,399,363A, March 1995, Liversidge et al.
U.S. Pat. No. 5,543,158A, August 1996, Gref et al.
U.S. Pat. No. 5,641,515A, June 1997, Ramtoola
U.S. Pat. No. 8,778,328 B2, July 2014, Erbs et al.
U.S. Pat. No. 8,980,246 B2, May 2015, Kim
U.S. Pat. No. 8,986,674 B2, March 2015, Kim and Thome
U.S. Pat. No. 9,005,602 B2, April 2015, Szalay et al.

1. A recombinant vector comprising:
a) a left flanking portion of a poxvirus gene selected from N1L, K1L, K3L, A46R, and A52R gene, and
b) a right flanking portion of said poxvirus gene,
wherein the left flanking portion and the right flanking portion are operably linked to a detectable interrupter expression cassette

2. (canceled)

3. The recombinant vector of claim 1, wherein the detectable interrupter expression cassette directs the expression of one or more polypeptides, selected from a fluorescent protein, a luciferase enzyme or a xanthine-guanine phosphoribosyltransferase (gpt) protein.

4. (canceled)

5. The recombinant vector of claim 1, wherein each flanking portion comprises about 200-600 nucleotide residues, preferably about 300-500 nucleotide residues, of flanking sequence and/or wherein he flanking sequence has at least 80%, at least 85, at least 90%, or at least 95% sequence identity to corresponding sequence in a poxvirus, optionally Accession number: NC_006998.1.

6. (canceled)

7. A recombinant oncolytic poxvirus or viral DNA thereof, constructed using the recombinant vector of claim 1.

8. A cell constructed with the recombinant vector of claim 1, optionally transfected with said recombinant vector or infected with a poxvirus, preferably a vaccinia virus, constructed with said recombinant vector or comprising viral DNA thereof.

9. A recombinant oncolytic poxvirus, optionally a recombinant oncolytic vaccinia virus or viral DNA thereof, comprising one or more inactivated poxvirus genes selected from N1L, K1L, K3L, A46R, and/or A52R, preferably selected from K1L, A46R and/or A52R, optionally further comprising an inactivated TK gene and/or one or more inactivated growth factor genes, optionally VGF genes.

10. The recombinant oncolytic poxvirus of claim 9, wherein the inactivated poxvirus genes are inactivated by one or more mutations, optionally deletion mutation comprising deletion of all or a portion of the gene or inactivated by replacing the poxvirus genes with a detectable interrupter expression cassette, optionally wherein the detectable interrupter expression cassette comprises one or more fluorescent proteins, luciferase enzymes or gpt proteins.

11. (canceled)

12. The recombinant oncolytic poxvirus or viral DNA thereof of claim 9, constructed using a recombinant vector, wherein the recombinant vector comprises:
a) a left flanking portion of a poxvirus gene selected from N1L, K1L, K3L, A46R, and A52R gene, and
b) a right flanking portion of said poxvirus gene,
wherein the left flanking portion and the right flanking portion are operably linked to a detectable interrupter expression cassette.

13. The recombinant oncolytic poxvirus or viral DNA thereof of claim 9, constructed using a parental strain selected from Lister, Wyeth, modified vaccinia Ankara, CV-1, Western Reserve, Copenhagen, Tian Tian and VJS6.

14. A cell infected by the oncolytic recombinant poxvirus or comprising viral DNA thereof of claim 9.

15. The cell of claim 14, wherein the cell is a tumour cell, optionally an ovarian cancer cell, a colorectal cancer cell, a hepatocellular carcinoma cell, a lung cancer cell, a mesothelioma cell, a prostate cancer cell, a melanoma cell, a renal cell carcinoma cell, a head and neck cancer cell, a pancreatic cancer cell, a glioma cell, a gastric cancer cell and/or a breast cancer cell.

16. A composition comprising the recombinant vector of claim 1, a cell or recombinant oncolytic poxvirus constructed using said recombinant vector, or viral DNA thereof and a suitable diluent, optionally a pharmaceutically suitable diluent.

17. (canceled)

18. An in vitro method of making the recombinant oncolytic poxvirus or viral DNA thereof of claim 9, comprising:
a) introducing a recombinant vector comprising:
i) a left flanking portion of a poxvirus gene selected from N1L, K1L, K3L, A46R, and A52R gene, and
ii) a right flanking portion of said poxvirus gene,
into cells infected with a poxvirus, optionally vaccinia virus, under conditions suitable for recombination between the recombinant vector and the recombinant oncolytic poxvirus or viral DNA; and
b) isolating the oncolytic poxvirus or viral DNA inactivated for the poxvirus gene selected from N1L, K1L, K3L, A46R, and A52R.

19. A method of killing cancer cells, comprising contacting the cancer cells or administering to a subject with a cancer or a tumour comprising cancer cells, an effective amount of the recombinant oncolytic poxvirus or viral DNA thereof, of claim 9.

20. The method of claim 19, wherein the cancer cells are solid tumour cells, optionally selected from ovarian cancer cells, colon cancer cells, hepatocellular carcinoma cells, lung cancer cells, mesothelioma cells, prostate cancer cells, melanoma cells, renal cell carcinoma cells, head and neck cancer cells, pancreatic cancer cells, glioma cells, gastric cancer cells, lymphoma cells and breast cancer cells, wherein the cancer cells are blood cancer cells, optionally myeloma cells or leukemic cells, or wherein the cancer cells are blood tumour cells, optionally lymphoma cells.

21. The method of claim 19, wherein the subject has late stage cancer, optionally having peritoneal carcinomatosis (PC).

22. The method of claim 19, wherein the subject is human.

23. An isolated DNA molecule used for amplifying flanking regions of a poxvirus gene selected from N1L, K1L, K3L, A46R and A52R, optionally the isolated DNA molecule selected from SEQ ID NOs:1-20 or an isolated DNA molecule comprising 5′ and/or 3′ overhang and sequence of flanking portion of a poxvirus gene selected from N1L, K1L, K3L, A46R and A52R, wherein the sequence amplified using a primer pair, optionally the primer pair selected from SEQ ID NOs:1-20.

24. (canceled)

25. A pharmaceutical composition comprising the recombinant oncolytic poxvirus of claim 9.

26. (canceled)

27. The pharmaceutical composition of claim 25 comprising a recombinant vaccinia virus.
